SEQUENCE LISTING (57 sequences)

SEQ ID NO: 1 - protein, 585 aa, Homo sapiens [sequence omitted]

SEQ ID NO: 2 - protein, 214 aa, Artificial Sequence
  Mutated/artificial sequence
  MISC_FEATURE: Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 451 aa, Artificial Sequence
  Mutated/artificial sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 1041 aa, Artificial Sequence
  Mutated/artificial sequence
  MISC_FEATURE: Trastuzumab heavy chain-HSA fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 213 aa, Artificial Sequence
  Mutated/artificial sequence
  MISC_FEATURE: Light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 451 aa, Artificial Sequence
  Mutated/artificial sequence
  MISC_FEATURE: Heavy Chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 1041 aa, Artificial Sequence
  Mutated/artificial sequence
  MISC_FEATURE: Rutuximab heavy chain-HSA fusion protein
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 214 aa, Artificial Sequence
  Mutated/artificial sequence
  MISC_FEATURE: LIght Chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, Artificial Sequence
  Mutated/artificial sequence
  MISC_FEATURE: Heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Mutated/artificial sequence
  MISC_FEATURE: Light chain
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 445 aa, Artificial Sequence
  Mutated/artificial sequence
  MISC_FEATURE: Heavy chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 1034 aa, Artificial Sequence
  Mutated/artificial sequence
  MISC_FEATURE: Panitumumab heavy chain-HSA fusion protein
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - protein, 218 aa, Artificial Sequence
  Mutated/artificial sequence
  MISC_FEATURE: Light chain
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 446 aa, Artificial Sequence
  Mutated/artificial sequence
  MISC_FEATURE: Heavy chain
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 446 aa, Artibeus aztecus; identical to SEQ ID NO: 14
  MISC_FEATURE: Brentuximab (XCD30) heavy chain-HSA fusion protein

SEQ ID NO: 16 - protein, 31 aa, Artificial Sequence
  Mutated/artificial sequence
  MISC_FEATURE: Human analog
HGEGTFTSDVSSYLEEQAAKEFIAWLVKGGG

SEQ ID NO: 17 - protein, 39 aa, Homo sapiens
  MISC_FEATURE: Peptide
HGEGTFTSDLSKQMEEEAVRLFIEWLKNGGPSSGAPPPS

SEQ ID NO: 18 - protein, 29 aa, Homo sapiens
  MISC_FEATURE: Peptide
HSQGTFTSDYSKYLDSRRAQDFVQWLMNT

SEQ ID NO: 19 - protein, 42 aa, Homo sapiens
  MISC_FEATURE: Peptide
YAEGTFISDYSIAMDKIHQQDFVNWLLAQKGKKNDWKHNITQ

SEQ ID NO: 20 - protein, 37 aa, Homo sapiens
  MISC_FEATURE: Peptide
HSQGTFTSDYSKYLDSRRAQDFVQWLMNTKRNRNNIA

SEQ ID NO: 21 - protein, 29 aa, Artificial Sequence
  Mutated/artificial sequence
  MISC_FEATURE: X is 2-aminoisobutyric acid, E at position 16 and K at position 20 form a ring
  misc_feature (2)..(2): Xaa can be any naturally occurring amino acid
HXQGTFTSDYSKYLDEKRAKEFVQWLMNT

SEQ ID NO: 22 - protein, 29 aa, Artificial Sequence
  Mutated/artificial sequence
  MISC_FEATURE: X is 2-aminoisobutyric acid, E at position 12 and K at position 16 form a ring.
  misc_feature (2)..(2): Xaa can be any naturally occurring amino acid
HXQGTFTSDYSKYLDEKRAKEFVQWLMNT

SEQ ID NO: 23 - protein, 39 aa, Artificial Sequence
  Mutated/artificial sequence
  MISC_FEATURE: X at position 2 and 29 is 2-isoaminobutyric acid, X at position 38 is C, C-PEG or absent, X at position 39 is C or absent, and when X at position 39 is a C it is optionally amidated
  misc_feature (2)..(2): Xaa can be any naturally occurring amino acid
  misc_feature (29)..(29): Xaa can be any naturally occurring amino acid
  misc_feature (38)..(39): Xaa can be any naturally occurring amino acid
HXQGTFTSDYSKYLDSKKAQEFVQWLLNXGRNRNNIAXX

SEQ ID NO: 24 - protein, 30 aa, Artificial Sequence
  Mutated/artificial sequence
  MISC_FEATURE: X2=S,2-aminoisobutryic acid; 3=E,Q;10=Y,V,L;12=K,S,I;13=L,Y,A;14=L,M;15=E,D;16=E,G,K;17=E,Q,I; 18=A,H;19=V,A,Q;20=R,K,Q;21=L,E,D;23=L,V;24=E,A,N; 27=L,K,V;28=A,K,N;29=G,Q;30=none,G,N,P,K,T
  misc_feature (2)..(3): Xaa can be any naturally occurring amino acid
  misc_feature (10)..(10): Xaa can be any naturally occurring amino acid
  misc_feature (12)..(21): Xaa can be any naturally occurring amino acid
  misc_feature (23)..(24): Xaa can be any naturally occurring amino acid
  misc_feature (27)..(30): Xaa can be any naturally occurring amino acid
YXXGTFTSDXSXXXXXXXXXXFXXWLXXXX

SEQ ID NO: 25 - protein, 29 aa, Artificial Sequence
  Mutated/artificial sequence
  MISC_FEATURE: X at position 2 is 2-aminoisobutyric acid, E at position 16 and K at position 20 for a ring
  misc_feature (2)..(2): Xaa can be any naturally occurring amino acid
HXQGTFTSDYSKYLDEQAAKEFICWLMNT

SEQ ID NO: 26 - protein, 34 aa, Homo sapiens
  MISC_FEATURE: Peptide
SVSEIQLMHNLGKHLNSMERVEWLRKKLQDVHNF

SEQ ID NO: 27 - protein, 34 aa, Artificial Sequence
  Mutated/artificial sequence
  MISC_FEATURE: X is 2-aminoisobutyric acid
  misc_feature (29)..(29): Xaa can be any naturally occurring amino acid
AVSEHQLLHDKGSIQNDLRRRELLEKLLXKLHTA

SEQ ID NO: 28 - protein, 216 aa, Artificial Sequence
  Mutated/artificial sequence
  MISC_FEATURE: Light Chain
QLVLTQSPSASASLGASVKLTCTLSSGHSNYAIAWHQQQPGKGPRYLNKVNRDGSHIRGDGIPDRFSGSTSGAERYLTISSLQSEDEADYYCQTWGAGIRVFGGGTKLTVLGAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 455 aa, Artificial Sequence
  Mutated/artificial sequence
  MISC_FEATURE: Heavy Chain
QVQLQKSGPGLVKPSETLSLTCTVSGGSISGYYWSWIRQPPGKGLEWIGYIHYSRSTNSNPALKSRVTISSDTSKNQLSLRLSSVTAADTAVYYCARDTYYYDSGDYEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 1045 aa, Artificial Sequence
  Mutated/artificial sequence
  MISC_FEATURE: F598 IgG1 heavy chain-HSA fusion protein
QVQLQKSGPGLVKPSETLSLTCTVSGGSISGYYWSWIRQPPGKGLEWIGYIHYSRSTNSNPALKSRVTISSDTSKNQLSLRLSSVTAADTAVYYCARDTYYYDSGDYEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 215 aa, Artificial Sequence
  Mutated/artificial sequence
  MISC_FEATURE: Light chain
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 448 aa, Artificial Sequence
  Mutated/artificial sequence
  MISC_FEATURE: Heavy chain
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 1038 aa, Artificial Sequence
  Mutated/artificial sequence
  MISC_FEATURE: Denosumab heavy chain-HSA fusion protein
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 34 - protein, 1038 aa, Artificial Sequence; identical to SEQ ID NO: 33
  Mutated/artificial sequence
  MISC_FEATURE: Light chain

SEQ ID NO: 35 - protein, 441 aa, Artificial Sequence
  Mutated/artificial sequence
  MISC_FEATURE: Heavy chain
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 1031 aa, Artificial Sequence
  Mutated/artificial sequence
  MISC_FEATURE: PCK9 heavy chain-HSA fusion protein
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 37 - protein, 214 aa, Artificial Sequence
  Mutated/artificial sequence
  MISC_FEATURE: Light chain
DIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 454 aa, Artibeus phaeotis
  MISC_FEATURE: Heavy chain
QVQLVESGGGVVQPGRSLRISCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDQSFFLYSKLTVDKSRWQQGNVFSCSVMHEALHNHYTQKSLSLSPKG

SEQ ID NO: 39 - protein, 1044 aa, Artificial Sequence
  Mutated/artificial sequence
  MISC_FEATURE: Glucagon receptor antibody heavy chain-HSA fusion protein
QVQLVESGGGVVQPGRSLRISCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDQSFFLYSKLTVDKSRWQQGNVFSCSVMHEALHNHYTQKSLSLSPKGGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 40 - protein, 1045 aa, Artificial Sequence
  Mutated/artificial sequence
  MISC_FEATURE: GPC antibody heavy chain-HSA fusion protein
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 41 - protein, 1051 aa, Artificial Sequence
  Mutated/artificial sequence
  MISC_FEATURE: GPC antibody heavy chain-HSA fusion proteins with two cysteine mutations in the HSA
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGCGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGDC

SEQ ID NO: 42 - protein, 1051 aa, Artificial Sequence; identical to SEQ ID NO: 41
  Mutated sequence

SEQ ID NO: 43 - DNA, 720 bp, Artificial Sequence
  Mutated/artificial sequence
  misc_feature: Light chain
atgcgcctccccgctcaacttcttggacttctcatgctctgggtgtcaggctcctccggagatgtcgtgatgacacagtcccctctctcactgcccgtgacccccggcgaaccggcttctatctcctgccggtcgtcccaatccctggtgcactcgaacgccaacacttatctgcattggtacctccaaaagccagggcagagcccccagctcctgatctacaaagtgtccaacaggttctccggcgtgccggatagattctccggatcggggagcggaaccgacttcacgctgaagatcagccgcgtcgaggccgaggacgtgggagtgtactactgcagccagaacacccacgtgccaccgactttcggacaagggaccaagctggagattaagcgtacggtggccgccccttccgtgtttatcttcccgccctcggacgaacagctgaagtccggtactgcctccgtcgtgtgcctgctgaacaacttctacccgcgcgaggccaaagtgcagtggaaggtcgataacgcgctgcagtccggcaacagccaggaaagcgtcaccgaacaggactccaaggactccacctactcgctgagcagcaccttgactctgtcgaaggccgactacgagaagcacaaggtctacgcgtgcgaagtgacccatcagggactgtcctcacctgtgaccaagtccttcaatcggggcgaatgttag

SEQ ID NO: 44 - DNA, 3195 bp, Artificial Sequence
  Mutated/artificial sequence
  misc_feature: GPC antobody heavy chain-HSA fusion
atggactggacttggagagtgttctgccttctcgccgtcgcccccggagcccattcgcaagtgcagctggtgcagtccggcgccgaagtcaagaagcccggtgcttcagtgaaagtgtcgtgcaaggcctccgggtacactttcaccgattacgaaatgcactgggtccgccaggcaccgggccagggcctggagtggatgggcgccctggaccccaagactggcgacaccgcgtattcgcagaagtttaagggccgcgtcactctcactgccgacaagtccactagcactgcgtacatggagctgtcatcactgaccagcgaggacaccgccgtgtactactgcactaggttctactcatacacctattggggacaaggcacccttgtcaccgtgtcctctgctagcactaagggaccttcggtgttcccactggccccgtcgtccaagtccacctcggggggaaccgcggccctgggatgcctcgtgaaggattacttcccggaacctgtcactgtgtcatggaacagcggagcactgacctccggggtgcacaccttccccgccgtgttgcagtccagcggtctttactccctgtcctccgtggtcaccgtcccctcctcctccctgggaacccagacttacatctgcaacgtcaaccacaagccgtccaacactaaggtcgacaagaaggtcgaacccaagtcctgtgacaagacgcacacctgtcctccatgccccgcacccgaactgctggggggtccgtccgtctttctcttcccgccgaagccgaaggacaccctgatgatctcaagaactccagaagtcacttgcgtggtcgtggatgtgtcccacgaggatcccgaagtgaagttcaattggtacgtggacggagtggaagtgcacaacgccaagaccaagcctcgggaagaacagtacaactccacctaccgggtggtgtccgtgctgaccgtcctgcaccaagactggctgaatgggaaggagtacaagtgcaaagtctccaacaaggcgctgccggccccaatcgagaaaactatcagcaaggccaaggggcagccccgggaacctcaagtgtacaccctccctccatcccgcgaagaaatgaccaagaaccaagtgtcgctgacttgccttgtgaagggcttctacccgtcggatatcgcggtggaatgggagtccaacggccagcctgaaaacaactacaagaccactccgccggtccttgactccgacggctccttcttcttgtactccaagctgaccgtggacaagagccggtggcagcagggcaacgtgttcagctgcagcgtgatgcacgaggccctgcataaccactatacccagaaaagcctgtccctgtcccctggaaagggagggggcggatcggggggtggaggctccggcggaggaggatcggacgcccacaaatcagaagtggcgcaccgattcaaagacctgggagaggagaacttcaaggcactcgtgcttattgcgttcgctcagtatctgcagcagtgcccgttcgaggaccacgtgaagctggtcaacgaagtcaccgaattcgcaaagacctgtgtcgccgatgaaagcgccgaaaactgcgacaagtccctgcacaccctgttcggagacaagctgtgcactgtggcgactctgcgcgaaacctacggagagatggccgactgttgcgctaagcaggaacccgaacgcaacgagtgcttcctgcaacacaaggatgacaatccaaacctcccccgcctcgtgcggcctgaagtcgacgtgatgtgcactgccttccacgacaacgaggaaaccttcttgaagaagtacctgtacgagatcgctcggcggcatccgtacttctacgccccggaactcctgttcttcgcgaagcggtacaaggcagcctttaccgagtgctgtcaagccgcggacaaggccgcttgtctgctgccaaagctggatgagctgcgggatgagggcaaagcctcctcagctaagcagcggctgaagtgcgccagcctgcagaaattcggcgaacgcgccttcaaggcctgggctgtcgcccggctgtcccaacggttccctaaggctgagtttgcggaagtgtcaaagctggtcactgacctcaccaaggtgcacactgaatgctgtcacggcgaccttctggagtgcgccgacgacagagccgacctggccaagtacatttgcgagaaccaggactcgatctcatccaagctcaaggagtgttgcgagaagccgctcctggagaaaagccactgcatcgctgaagtggagaacgacgaaatgcccgcagacttgccttcgctcgccgcggacttcgtggaatctaaggacgtgtgcaagaactatgcagaggccaaggacgtgtttctcggaatgttcctgtacgaatacgcgaggcgccaccctgattactcagtggtgctgctgctgcgcctcgccaagacctacgaaaccacactggaaaagtgttgtgccgctgccgatccgcacgagtgttacgcgaaggtgttcgatgagttcaagccgttggtggaggagccgcagaacctgattaagcagaactgcgaactgtttgaacagctcggagaatacaaattccagaacgccctgctcgtgcggtacactaagaaagtgccgcaagtgtcgaccccaaccctggtggaagtgtcgagaaatttgggaaaagtcggatccaaatgctgcaagcatcctgaggccaagaggatgccttgcgccgaggattacctgagcgtggtgctgaaccagctttgcgtgttgcatgaaaagacccccgtgtccgaccgcgtgactaagtgttgcaccgagtccctcgtgaatcgccggccatgttttagcgctctggaggtggacgagacttacgtgcccaaggagttcaacgccgaaaccttcaccttccacgccgacatctgcacgctgagcgagaaggagcggcagattaagaagcagaccgccctcgtggaactcgtgaagcataagcctaaggccaccaaggagcagctgaaggcggtcatggacgacttcgcggcatttgtggagaagtgttgcaaggccgacgacaaagaaacgtgcttcgccgaagaaggaaagaagttggtggccgccagccaggccgctctcgggctgtag

SEQ ID NO: 45 - DNA, 3195 bp, Artificial Sequence; identical to SEQ ID NO: 44
  Mutated/artificial sequence
  misc_feature: GPC antibody heavy chain-HSA fusion with mutations

SEQ ID NO: 46 - protein, 5 aa, Artificial Sequence
  Mutated/artificial sequence
  MISC_FEATURE: CDR
NYGMN

SEQ ID NO: 47 - protein, 17 aa, Artificial Sequence
  Mutated/artificial sequence
  MISC_FEATURE: CDR
WINTYTGEPTYTDDFKG

SEQ ID NO: 48 - protein, 10 aa, Artificial Sequence
  Mutated/artificial sequence
  MISC_FEATURE: CDR
GGYDGRGFDY

SEQ ID NO: 49 - protein, 10 aa, Artificial Sequence
  Mutated/artificial sequence
  MISC_FEATURE: CDR
SASSSVSYMH

SEQ ID NO: 50 - protein, 7 aa, Artificial Sequence
  Mutated/artificial sequence
  MISC_FEATURE: CDR
DTSKLAS

SEQ ID NO: 51 - protein, 9 aa, Artificial Sequence
  Mutated/artificial sequence
  MISC_FEATURE: CDR
QQWSGYPLT

SEQ ID NO: 52 - protein, 5 aa, Artificial Sequence
  Mutation/artificial sequence
  MISC_FEATURE: CDR
GYTMN

SEQ ID NO: 53 - protein, 17 aa, Artificial Sequence
  Mutation/artificial sequence
  MISC_FEATURE: CDR
LITPYNGASSYNQKFRG

SEQ ID NO: 54 - protein, 10 aa, Artificial Sequence
  Mutation/artificial sequence
  MISC_FEATURE: CDR
GGYDGRGFDY

SEQ ID NO: 55 - protein, 10 aa, Artificial Sequence
  Mutation/artificial sequence
  MISC_FEATURE: CDR
SASSSVSYMH

SEQ ID NO: 56 - protein, 7 aa, Artificial Sequence
  Mutation/artificial sequence
  MISC_FEATURE: CDR
DTSKLAS

SEQ ID NO: 57 - protein, 9 aa, Artificial Sequence
  Mutation/artificial sequence
  MISC_FEATURE: CDR
QQWSGYPLT

(19) United States
(12) Patent Application Publication
(10) Pub. No.: US 2018/0055944 A1
(43) Pub. Date: Mar. 1, 2018

(54) NOVEL ANTIBODY-ALBUMIN-DRUG CONJUGATES (AADC) AND METHODS FOR USING THEM

(71) Applicant: AskGene Pharma Inc., Camarillo, CA (US)

(72) Inventors: Yuefeng Lu, Newbury Park, CA (US); Jian-Feng Lu, Oak Park, CA (US); Lan Yang, Camarillo, CA (US); Lu Li, Camarillo, CA (US); Lei Liu, Thousand Oaks, CA (US); Shiwen Zhang, Camarillo, CA (US)

(73) Assignee: AskGene Pharma Inc., Camarillo, CA (US)

(21) Appl. No.: 15/666,515

(22) Filed: Aug. 1, 2017

Related U.S. Application Data
(63) Continuation of application No. PCT/US2017/044771, filed on Jul. 31, 2017.
(60) Provisional application No. 62/369,649, filed on Aug. 1, 2016.
(60) Provisional application No. 62/463,500, filed on Feb. 24, 2017.

(51) Int. Cl. A61K 47/64; A61K 47/68; C07K 16/30; C07K 14/765; A61K 38/07

(52) U.S. Cl. CPC A61K 47/643; A61K 47/6817; C07K 16/303; A61K 47/6859; C07K 14/765; C07K 2317/73; C07K 2319/31; C07K 2317/56; C07K 2317/77; C07K 2319/33; A61K 38/07

(57) ABSTRACT

The present invention relates to compositions and methods using an isolated chimeric molecule, wherein the isolated chimeric molecule comprises an antibody, one or more albumin motifs, optional peptide linkers, and two or more antibiotics or cytotoxic drug molecules conjugated to the unpaired cysteine residues, optionally through linkers. In one embodiment, each of the said albumin motifs in the isolated chimeric molecule contains 2 or more unpaired cysteine residues. In another embodiment, the said antibody in the isolated chimeric molecule targets antigens on cancer cells or drug-resistant bacteria. In another embodiment, the cancer cells have upregulated macropinocytosis. In another embodiment, the cancer cells contain one or more mutations in their RAS family genes. The compositions of the invention are used to treat drug-resistant bacterial infections and cancers, preferably the ones with upregulated macropinocytosis, and the ones containing one or more mutations in their RAS family genes.

### CROSS REFERENCE TO RELATED APPLICATIONS

The present application claims priority to U.S. provisional patent application 62/369,649, filed Aug. 1, 2016, U.S. provisional patent application 62/463,500, filed Feb. 24, 2017, and PCT/US2017/044771, filed Jul. 31, 2017, each herein incorporated by reference in their entirety.

### INTRODUCTION

Human Serum Albumin (HSA, HA): Albumin a protein of 585 amino acids in its mature form, is responsible for a significant proportion of the osmotic pressure of serum and also functions as a carrier of endogenous and exogenous ligands. It is the principal transport protein in human plasma. It is highly soluble and is the most abundant plasma protein in the blood, at 35-50 g/L (Kratz F. Albumin as a drug carrier: Design of prodrugs, drug conjugates and nanoparticles. J Control Release. 2008; 132(3):171-83). It binds a broad spectrum of compounds and metabolites, for example, steroids, bile acids, fatty acids and amino acids, heavy metals and pharmacological substances, for example warfarin. The substances bind primarily to two subdomains or hydrophobic pockets of the albumin, Sudlow I (bilirubin, warfarin) and Sudlow II (tryptophan, octanoate, fatty acids), with heavy metals mainly binding to the N-terminus (Peters et al., All about Albumin (1996) Academic Press). In addition, HSA contains a free thiol at C34, which is ideal to conjugate active drug molecules and peptides to it through a sulfhydryl reactive group such as maleimide. Human serum albumin At present, HA for clinical use is produced by extraction from human blood. The production of recombinant HSA (rHSA) in microorganisms has been disclosed in EP 330 451 and EP 361 991. A typical clinical use of albumin is as plasma volume expander. Within the last 10 years, however, albumin has found increasing therapeutic use as a transport protein on account of its physiological properties.

Albumin has been used as carrier for hydrophobic drug molecules. It was reported that each albumin molecule may be able to carry up to 4-6 hydrophobic molecules (Garro et al. 2011, Int. J Nanomedicine. 2011; 6: 1193-1200). Conjugation of hydrophobic drug molecules to the albumin motifs would shield the hydrophobicity of the drug molecules.

A number of mutations to the albumin molecule have been made, in order to increase its half-time in plasma, change its binding characteristics to metal ions, or increase the number of conjugation competent cysteine residues. It was disclosed in patent publication WO2013/075066, wherein the albumin variants had longer half-lives than the natural one. In U.S. Pat. No. 6,787,636, the albumin molecule was engineered to reduce its binding affinities to metal ions such as copper and nickel.

Patent publication WO2010/092135 disclosed “thiol-albumins” wherein the mutated albumin contains two or more conjugation competent cysteine residues. Specifically, the human serum albumin with sequence such as SEQ ID NO:1 comprises one or more of:
(a) substitution of a non-cysteine amino acid with a cysteine at a position corresponding to a position equivalent to any of residues L585, D1, A2, D562, A364, A504, E505, T79, E86, D129, D549, A581, D121, E82, S270, Q397 and A578 of SEQ ID NO:1;
(b) insertion of a cysteine at a position adjacent the N- or C-side of an amino acid which may or may not correspond to a position equivalent to any of residues L585, D1, A2, D562, A364, A504, E505, T79, E86, D129, D549, A581, D121, E82, S270, Q397 and A578 of SEQ ID NO:1;
(c) a cysteine with a free thiol group at a position which may or may not correspond to any of C369, C361, C91, C177, C567, C316, C75, C169, C124 and C558 which may or may not be generated by deletion or substitution of C360, C316, C75, C168, C558, C361, C91, C124, C169 and/or C567; and/or
(d) addition of a cysteine to the N-side of the N-terminal residue of an albumin sequence and/or to the C-side of the C-terminal residue of an albumin sequence such that the net result of the substitution, deletion, addition or insertion events of (a), (b), (c) and (d) is that the number of conjugation competent cysteine residues of the polypeptide sequence is increased relative to the polypeptide prior to the substitution, insertion, deletion and addition events.
Within (a) to (d), above, all of the residues are preferred. However, within each of (a), (b), (c) and (d), the residues are listed in order of decreasing preference. A thio-albumin may or may not include a polypeptide where one or more naturally occurring free-thiol group(s), such as cysteine-34 in HSA (SEQ ID NO:1), is modified to an amino acid which is not cysteine. For example, cysteine may or may not be replaced by an amino acid which has a relatively high conservation score such as alanine or serine. A thio-albumin may or may not include a poly-peptide where one or more naturally occurring free-thiol group(s), such as cysteine-34 in HSA (SEQ ID NO:1) are present.

Antibiotics:

Multidrug resistance microbial infection is an urgent unmet medical need. It is a crisis at the global level. A number of antibodies have been developed targeting multidrug resistance microbial infections. Unfortunately, few antibodies have reached commercialization. In fact, many of them already failed in clinical trials, mainly due to insufficient efficacy

MRSA is methicillin-resistant Staphylococcus aureus, a potentially dangerous type of staph bacteria that is resistant to certain antibiotics and may cause skin and other infections. It is estimated that Americans of all ages visit the doctor more than 12 million times per year for skin infections that are typical of staph, more than half of which are MRSA. It was estimated that MRSA costs eight billion dollars for hospital stays annually and has killed 20,000-40,000 people in 2007 in USA.

Many MRSA infections still can be treated by certain specific antibiotics (for example, vancomycin (VANCOCIN®), linezolid (ZYVOX®), and others, often in combination with vancomycin). Most moderate to severe infections need to be treated by intravenous antibiotics, usually given in the hospital setting. Some CA-MRSA strains are susceptible to trimethoprim sulfamethoxazole (BACTRIM®), doxycycline (VIBRAMYCIN®), and clindamycin (CLEOCIN®); although reports suggest clindamycin resistance is increasing rapidly. In addition, some strains are now resistant to vancomycin. In 2011, researchers developed a chemical change in the antibiotic vancomycin that rendered vancomycin-resistant MRSA susceptible to the drug. It is not available commercially, but this discovery, along with ongoing research, is important because it may expand treatment possibilities for MRSA and other drug-resistant bacteria such as VRE (vancomycin-resistant enterococci). Another drug, ceftaroline fosamil (TEFLARO®), has been approved for treatment by the FDA for MRSA infections.

A note of caution is that, in the last few years, there have been reports of a new strain of MRSA that is resistant to vancomycin (VRSA or vancomycin-resistant S. aureus) and other antibiotics. Currently, VRSA is detected more often than a few years ago, but if it becomes widespread, it may be the next superbug.

In addition to antibiotics, a number of antibodies and vaccines have been tried or are currently in development for treatment of MRSA infections (Table 1). Those antibodies and vaccines have been mainly targeting Microbial Surface Component Recognizing Adhesive Matrix Molecules (MSCRAMM), which include CIfA, ClfB, SdrC, SdrD, and SdrE, among others. Unfortunately, most of the programs failed expensive failures in late stage clinical development mainly because of lacking of efficacy. At least some of the leading scientists in the field attributed the failure to the lacking of T cell stimulation (McKenna, Nature 482:23-25 (2012), doi:10.1038/482023a).

TABLE 1
Antibody/vaccine drug candidates for MRSA

| Drug Candidate | Company | Target | Status |
|---|---|---|---|
| Aurexis- | Inhibitex | ClfA | Completed phase II; |
| Humanized Mab | | | project suspended |
| Aurograb ScFv | NeuTec | ABC | Phase III ended in |
| | (Aquired | Transporter | 2006; failed |
| | by Novatis) | | efficacy |
| Pagibaximab | Biosynexus | Lipoteioic | Phase III failed |
| | | Acid | efficacy |
| V710 (Vaccine) | Merck | Iron surface | Phase III failed |
| | | determinant B | efficacy |
| F598 | Alopexx | PNAG-Poly-N- | Moving into Phase |
| | (Licensed | Acetyl- | III |
| | by Sonafi) | Glucosamine | |

In addition to MRSA infections, infections caused by other bacteria, fungi, protozoa, multi-cellular parasites, and viruses such as hepatitis B virus (HBV), hepatitis C virus (HCV), human immunodeficiency virus (HIV), and human papillomavirus (HPV) are also serious and sometimes devastating, especially in developing countries.

Antibody-Drug Conjugates (ADC):

Antibody-drug conjugates (ADC) have been intensively researched as therapeutics to treat cancers. The drug molecules are selected from chemotherapeutic agents, which are often hydrophobic. To this date, the ADC molecules are typically formed by conjugating the drug molecules, through a linker, to amine groups present in the antibody molecule or free thiol groups introduced into the antibody molecule through engineering. The number of drug molecules that are able to be conjugated to a given antibody molecule (the drug/antibody ratio, or DAR) is limited, typically no more than four per antibody, at least in part due to the hydrophobicity of the drug molecules, which would destabilize the antibody molecule if the number of drug molecules per antibody is more than four. There are situations when the density of the target molecules on cancer cell surface are limited, and consequently a higher DAR is required in order to achieve therapeutic efficacy.

Most of the ADC molecules in clinical trials have half-lives of about 7 days or shorter (Antoine Deslandes, mAbs 6:4, 859-870, 2014), which were in general significantly shorter than the naked antibodies. While the loss of the drugs from antibody-drug conjugates over time contributed significantly to the reduced half-lives, the hydrophobicity of the conjugates because of the drug molecules may have also contributed significantly to the reduction of half-lives in vivo (Lyon et al., “Reducing hydrophobicity of homogeneous antibody-drug conjugates improves pharmacokinetics and therapeutic index”. Nature Biotechnology 33, 733-735 (2015)). It was reported that “hydrophobicity and plasma clearance also correlated with the hepatic uptake of these ADCs” (Lyon et al., 2015). In addition, the increased exposure also translated into higher therapeutic efficacy as demonstrated in the xenograft animal model (Lyon et al., 2015).

Anti-Cancer Drug Molecules:

Majority of the cytotoxic molecules used in ADC are microtubule disrupting agents and DNA modifying agents. Additional cytotoxic agents include RNA polymerase inhibitors, and topoisomerase I inhibitors. Examples of drug molecules frequently used in the ADC setting are reviewed by Kim & Kim, BioMol Ther (Seoul) 2015 November; 23(6): 493-509.). The drug molecule in the ADC setting can be selected, for example, from the following: azaribine, anastrozole, azacytidine, bleomycin, bortezomib, bryostatin-1, busulfan, calicheamicins, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, duocarmycin, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, monomethyl auristatin E (MMAE), monomethyl auristatin F (MMAF), phenylbutyrate, prednisone, procarbazine, paclitaxel, pentostatin, pyrrolobenzodiazepine (PBD), semustine, SN-38, streptozocin, tamoxifen, taxanes, taxol, testosterone propionate, thalidomide, thioguanine, thiotepa, teniposide, topotecan, uracil mustard, vinblastine, vinorelbine, vincristine.

Active Peptides:

Many peptides have therapeutic effects. Some of them have been developed into effective therapies or are in late stage clinical development, such as Exendin, GLP-1 analogs, PTH analog and PTHrP analog. In order to overcome the susceptibility to protease degradation in vivo, a number of the peptide drug candidates in development contain unnatural amino acids. Dual agonists are in development, such as oxyntomodulin, GLP-1/GIP, GLP-1/Glucagon. Many of them contain unnatural amino acids. Tri-agonists are also in development.

Peptides also have short half-life in vivo. There is a need to fuse or conjugate the peptides to a carrier such as albumin, Fc, and antibodies in order to extend their half-lives in vivo. A number of combinations, such as PTH analog with Denosumab, or GLP-1 or oxyntomodulin analogs with PCSK9 antibodies, may provide synergy in therapeutic efficacy while adding convenience in delivery.

Linkers:

In general, the drug molecules attached to the linkers, which further conjugate to free amine or free thiol groups on the antibody of the ADC. Examples of linkers used in ADC application have been disclosed in U.S. Pat. No. 7,754,681. Linker selections, chemistry and ADC examples have also been described by Jain et al (Pharm Res. 2015; 32(11): 3526-3540).

Linkers used in ADC include cleavable ones and non-cleavable ones. Principally, cleavable linkers exploit the differences in intracellular pH, reduction potential or enzyme concentration to trigger the release of the cytotoxin in the cell. Non-cleavable linkers do not contain release mechanism. ADCs with non-cleavable linkers rely on the complete degradation of the antibody after internalization of the ADC to release the payloads.

The linker used in the ADC technology needs to be stable in plasma after drug administration for an extended period of time such that the ADC can localize to tumor cells. While inside the cancer cells, the payload needs to be released so that the payloads can exercise their cell killing functionality. Premature release of the payloads in plasma leads to toxicity and lowering down the therapeutic index of the ADCs. In addition, linkers can have a profound effect on the characteristics and stability of ADC molecules. ADC aggregates may be formed due to the hydrophobicity of the drug molecules, which may lead to reduced stability and higher immunogenicity. In addition, the linker may impact the hydrophobic characteristic of the drug molecule. For example, Kovtun et al. has shown that using a hydrophilic linker with maytansinoids produced a hydrophilic metabolite of DM1 which was not an MDR substrate (Cancer Res. 2010; 70:2528-37). This hydrophilic-linked DM1 ADC was markedly more effective in an MDR-1-expressing human xenograft tumor than the MCC-DM1 ADC.

Maleimides are commonly used to attached the drug molecules to the thiol groups of proteins and antibodies. The formed conjugates may be instable and cleaved in vivo due to reactions such as thiol exchange. The cysteine-linked antibody drug conjugates may be stabilized using N-aryl maleimides (Christie et al., J Control Release, 2015 Dec. 28; 220(Pt B):660-70). Conjugates made with electron-withdrawing maleimides can purposefully hydrolyzed to their ring-opened counterparts in vitro to ensure in vivo stability (Fontaine et al., Bioconjug Chem, 2015 Jan. 21; 26(1):145-52). ADC created with these chemistries had significantly higher stability and reduced loss or premature release of the payloads from the antibody-drug conjugates in plasma.

Antibodies and Targets for Antibody-Drug Conjugates (ADC):

Many antibodies against a large number of oncology targets are in development as ADC (Kim and Kim, Biomol Ther 23(6): 493-509 (2015)). Additional antibodies are emerging as potential targets for ADC-based treatments. The targets include: HER2, HER3, DLL-3, DLL-4, EGF Receptors, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, GCC, Mesothelin, 5T4, CD19, CD20, CD22, CD33, CD40, CD56, CD66e, CD70, CD74, CD79b, CD98, CD123, CD138 and CD352.

DLL-3 is usually an intracellular protein. It was found that it is also expressed on the surface of cancer cells (see, e.g., WO2014125273A1). A number of antibodies against DLL-3 have also been developed. They include MAB4315 (R&D Systems, Inc.) and the antibodies disclosed in EP 2530091. It has been demonstrated that MAB4315, DL308 and DL309 internalized once binding to the DSL domain and the EGF domain of DLL-3 on cancer cell surface. At least one DLL-3 antibody-drug conjugate (ADC) has been in development for lung cancer.

CMET antibodies have been in development for gastric cancers among other indications. A number of antibodies have been developed for CMET. Several of them have been developed as antibody-drug conjugates for cancer treatment (see examples described in U.S. Pat. No. 9,364,556 B2, and patent application WO2013003680 A1)

Glypican-3 is one of the glypican family of heparan sulfate proteoglycans expressed on cell surface. GPC-3 may also be cleaved at the 358th or 374th amino acid and secreted into blood. GPC-3 has been shown to be over expressed on cancer cells including liver cancer. A number of antibodies have been developed for GCP3. One of the GPC-3 antibodies has been in middle stage of clinical development for liver cancer. GCP3 antibodies have been disclosed in U.S. Pat. No. 7,919,086. For example, one of the antibodies GC33 bound to an epitope that is located closer to the C-terminal of the extracellular domain of the GPC-3 molecule, and is located after the cleavage sites.

Pancreatic and Colorectal Cancer Targets:

Many of the antigens on cancer cell surface, such as the ones shown in Table 2, are targets for antibody and ADC drugs to treat cancers including pancreatic and colorectal cancers.

The transmembrane cell surface receptor guanyl cyclase C (GCC) is expressed by ˜95% of primary and metastatic colorectal cancer (mCRC) tumours and in subsets of pancreatic cancers. GCC antibody-drug conjugates have been disclosed in patent application EP 2490720 A1.

Mesothelin has been suggested as a therapeutic target because it is highly expressed in malignant mesotheliomas (Chang et al., Cancer Res 52: 181-186, 1992; Chang and Pastan, Proc Natl Acad Sci USA 93: 136-140, 1996) and other solid tumors, such as stomach cancer, squamous cell carcinomas, prostate cancer, pancreatic cancer, lung cancer, cholangiocarcinoma, breast cancer and ovarian cancer (Hassan et al., Clin. Cancer Res. 10:3937-3942, 2004; McGuire et a.I, N. Engl. J. Med. 334: 1-6, 1996; Argani et al., Clin. Cancer Res. 7:3862-3868, 2001; Hassan et al., Appl. Immunohistochem. Mol. Morphol. 13:243-247, 2005; Li et al., Mol. Cancer Ther. 7:286-296, 2008; Yu et al., J Cancer 1: 141-1749, 2010; Tchou et al., Breast Cancer Res Treat 133(2):799-804, 2012; U.S. Pat. No. 7,081,518).

Antibodies targeting mesothelin have been disclosed in a number of patents or patent applications, including U.S. Pat. No. 8,206,710; U.S. Pat. No. 7,081,518; U.S. Pat. No. 7,592,426; U.S. Pat. No. 8,460,660; U.S. Pat. No. 9,409,992; and US patent publication 2015/0065388.

Trop-2 is another target for ADC cancer therapeutics (Goldenberg et al., Oncotarget 2015; 6(26):22496-512). It was expressed and associated with poor prognosis of a number of solid tumors, including pancreatic, gastric, ovarian, colorectal, breast, oral and lung cancers (Ambrogi et al., PLoS One 2014; 9(5): e96993). While it was also expressed in normal tissues, upregulation of Trop-2 gene was found to stimulate tumor growth (Trerotola et al., Oncogene 32, 222-233, 2013). Antibodies and ADCs targeting Trop-2 have been disclosed in a number of patents or patent applications, including: U.S. Pat. No. 7,420,040, U.S. Pat. No. 8,871,908, U.S. Pat. No. 9,062,100, and US Patent Publication 2014/0377287.

Other targets for pancreatic cancer treatment including:
A phase II study of MM-141, an antibody targeting HER3 and IGF-1R, plus chemotherapy in patients with front-line metastatic pancreatic cancer (NCT02399137).
A phase I trial of MVT-5873, an antibody against the carbohydrate antigen 19-9, in patients with pancreatic cancer (NCT02672917). CA19-9 is overexpressed on a number of different tumor cell types, and plays a key role in tumor cell survival and metastasis.

As shown in the Table 2 (from reference: Scott et al., Nature Reviews Cancer 12, 278-287 (April 2012)), a number of antigens can be targeted for colorectal cancer therapy, including CEA, EpCam, Mucins, EGFR, and gpA33.

TABLE 2

| | | Examples of | |
|---|---|---|---|
| | | therapeutic mAbs | |
| Antigen | Examples of | raised against | Tumour types |
| category | antigens | these targets | expressing antigen |
| Haematopoietic | CD20 | Rituximab | Non-Hodgkin's |
| differentiation | | | lymphoma |
| antigens | | Ibritumomab | Lymphoma |
| | | tiuxetan and | |
| | | tositumomab | |
| | CD30 | Brentuximab | Hodgkin's lymphoma |
| | | vedotin | |
| | CD33 | Gemtuzumab | Acute myelogenous |
| | | ozogamicin | leukaemia |
| | CD52 | Alemtuzumab | Chronic lymphocytic |
| | | | leukaemia |
| Glycoproteins | EpCAM | IGN101 and | Epithelial tumours |
| expressed by | | adecatumumab | (breast, colon and |
| solid tumours | | | lung) |
| | CEA | Labetuzumab | Breast, colon and |
| | | | lung tumours |
| | gpA33 | huA33 | Colorectal carcinoma |
| | Mucins | Pemtumomab and | Breast, colon, lung |
| | | oregovomab | and ovarian tumours |
| | TAG-72 | CC49 | Breast, colon and |
| | | (minretumomab) | lung tumours |
| | CAIX | cG250 | Renal cell carcinoma |
| | PSMA | J591 | Prostate carcinoma |
| | Folate-binding | MOv18 and | Ovarian tumours |
| | protein | MORAb-003 | |
| | | (farletuzumab) | |
| Glycolipids | Gangliosides | 3F8, ch14.18 and | Neuroectodermal |
| | (such as GD2, | KW-2871 | tumours and some |
| | GD3 and GM2) | | epithelial tumours |
| Carbohydrates | Le | hu3S193 and | Breast, colon, lung |
| | | IgN311 | and prostate tumours |
| Targets of anti- | VEGF | Bevacizumab | Tumour vasculature |
| angiogenic | VEGFR | IM-2C6 and | Epithelium-derived |
| mAbs | | CDP791 | solid tumours |
| | Integrin αVβ3 | Etaracizumab | Tumour vasculature |
| | Integrin α5β1 | Volociximab | Tumour vasculature |
| Growth and | EGFR | Cetuximab, | Glioma, lung, breast, |
| differentiation | | panitumumab, | colon, and head and |
| signaling | | nimotuzumab and | neck tumours |
| | | mAb806 | |
| | ERBB2 | Trastuzumab and | Breast, colon, lung, |
| | | pertuzumab | ovarian and prostate |
| | | | tumours |
| | ERBB3 | MM-121 | Breast, colon, lung, |
| | | | ovarian and prostate, |
| | | | tumours |
| | MET | AMG 102, | Breast, ovary and |
| | | METMAB and | lung tumours |
| | | SCH 900105 | |
| | IGF1R | AVE1642, IMC- | Glioma, lung, breast, |
| | | A12, MK-0646, | head and neck, |
| | | R1507 and | prostate and thyroid |
| | | CP 751871 | cancer |
| | EPHA3 | KB004 and IIIA4 | Lung, kidney and |
| | | | colon tumours, |
| | | | melanoma, glioma |
| | | | and haematological |
| | | | malignancies |
| | TRAILR1 | Mapatumumab | Colon, lung and |
| | | (HGS-ETR1) | pancreas tumours |
| | | | and haematological |
| | | | malignancies |
| | TRAILR2 | HGS-ETR2 and | |
| | | CS-1008 | |
| | RANKL | Denosumab | Prostate cancer and |
| | | | bone metastases |
| Stromal and | FAP | Sibrotuzumab | Colon, breast, lung, |
| extracellular | | and F19 | pancreas, and head |
| matrix antigens | | | and neck tumours |
| | Tenascin | 81C6 | Glioma, breast and |
| | | | prostate tumours |

EGFR Mutants:

Antibody-drug conjugates have been designed based on antibodies that target a mutated but naturally occurring version of EGFR, known as EGFRvIII, or on conformational forms of the EGFR, both of which predominate on tumor cells and not on skin cells, as described in U.S. Pat. No. 7,628,986, and U.S. Pat. No. 7,589,180. For another example, anti-EGFR antibody mAb806 is an antibody that targets an EGFR epitope found only on cancer cells, and potentially offers an advantage over the current EGFR antibodies, which all display significant binding to normal organs such as skin in humans, as described in U.S. Pat. No. 7,767,792.

Macropinocytosis and RAS Mutations: Macropinocytosis is a highly conserved endocytic process which results into internalization of large patches of plasma membrane along with extracellular fluid through macropinosomes (Comisso et al., Nature 497, 633-637; Ha et al, Front Physiol. 2016; 7: 381). Cancer cells may have upregulated marcropinocytosis in order to meet the nutrient requirements for their fast growth. Podocytes and colorectal cancer cells activate macropinocytosis through interactions between albumin-associated free fatty acids (FFAs) and GPCRs (Wu et al., Oncogene 32, 5541-5550, 2013; Chung et al., J. Clin. Invest. 125, 2307-2316, 2015). Stimulation of EGFR and oncogentic RAS expression can actively induce macropinocytosis in cancer cells (Narkase et al., Sci Report. 2015; 5: 10300).

The mammalian ras gene family comprises H-ras, K-ras, N-ras, encoding H-ras, K-ras, N-ras proteins, respectively, with a similar structure and function. The Ras protein is located in the inner region of the cell membrane, tranforms signals from EGFR to mitogen-activated protein kinases (MAPKs), to control cell growth, proliferation, and motility, as well as metastasis and angiogenesis (Kiaris H, Spandidos D A. Mutations of ras genes in human tumours. International Journal of Oncology. 1995; 7:413-429). The K-ras gene usually contains point mutations at codons 12, 13 and 61, and these mutations often activate the K-ras oncogene (Schubbert et al., Hyperactive Ras in developmental disorders and cancer. Nat Rev Cancer. 2007; 7:295-308; Bos et al. Prevalence of ras gene mutations in human colorectal cancers. Nature. 1987; 327:293-297). The K-ras mutation status is associated with the therapeutic efficacy of EGFR-targeting monoclonal antibodies, rendering patients with K-ras mutation as not suitable for Erbitux treatment (Benvenuti, et al. Oncogenic activation of the RAS/RAF signaling pathway impairs the response of metastatic colorectal cancers to anti-epidermal growth factor receptor antibody therapies. Cancer Res. 2007; 67:2643-2648).

Per the RAS Initiative of the National Institute of Cancer (NCI), more than 30% of all human cancers—including 95% of pancreatic cancers and 45% of colorectal cancers—are driven by mutations of the RAS family of genes. Among them, there were about 9.1%, 18.6% and 6.7% of K-ras mutations in lung, colorectal and gastric cancers, respectively (Peng and Zhao, Oncol Lett. 2014 August; 8(2): 561-565). However, attempts to develop anti-cancer treatments targeting mutated RAS proteins remained to be unsuccessful (Gysin et al., Genes Cancer 2011 March; 2(3): 359-372). Tumors bearing RAS mutations remain among the most difficult to treat. Among other changes, oncogenic Ras promotes glucose fermentation and glutamine use to supply central carbon metabolism (White, Genes & Dev. 2013. 27:2065-2071). It was also demonstrated by Commisso et al. (Nature 497, 633-637, 2013) that oncogenic RAS protein was able to stimulating the macropinocytosis of the cancer cells, a process the cancer cells utilized to obtain nutrients for rapid growth. As the most abundant plasma protein in the blood, albumin is the desired cargo for the macropinocytosis process.

Nanoparticles:

Anticancer nanoparticles are developed to capsuling hydrophobic drug molecules. Among them are albumin-based nanoparticles (e.g., albumin bound paclitaxel (ABRAXANE©) and liposome-based nanoparticles. For example, compositions and methods of making albumin-based nanoparticles have been disclosed in U.S. Pat. No. 8,846,771.

### SUMMARY OF THE INVENTION

In one aspect, the invention provides an isolated antibody-albumin fusion molecule, comprising an antibody and at least one albumin or albumin fragment (also referred as albumin motif), wherein the albumin motif is fused to the heavy chains and/or light chains of said antibody, optionally through a peptide linker, wherein the albumin motif is a human serum albumin variant, which is a mutant of human serum albumin, which has been mutated such that the mutant contains a total of two or more unpaired cysteine residues.

In one embodiment, the antibody is selected from the group consisting of a single-chain Fv antibody (scFv), a Fab antibody, a Fab′ antibody, a (Fab′)2 antibody, a domain antibody, a nanobody, a minibody, a maxibody, a diabody, and a bispecific antibody. In one embodiment, the antibody is selected from the group consisting of IgG1, IgG2 and IgG4, and wherein the antibody is a chimeric antibody with human constant domains, a humanized antibody, or a fully human antibody.

In one embodiment, the albumin variant has at least 90% sequence identity to the amino acid sequence of SEQ ID NO:1.

In one embodiment, each albumin motif contains one or more substitutions of non-cysteine residue to cysteine residue at a position selected from the group consisting of L585, D1, A2, D562, A364, A504, E505, T79, E86, D129, D549, A581, D121, E82, S270, Q397 and A578 of SEQ ID NO:1.

In one embodiment, each albumin motif contains one or more insertions of a cysteine residue at a position adjacent to the N- or C-side of an amino acid at a position selected from the group consisting of L585, D1, A2, D562, A364, A504, E505, T79, E86, D129, D549, A581, D121, E82, S270, Q397 and A578 of SEQ ID NO:1.

In one embodiment, the albumin variant contains of one or more free thiol groups at a position selected from the group consisting of C369, C361, C91, C177, C567, C316, C75, C169, C124 and C558, which are generated by deletion or substitution of C360, C316, C75, C168, C558, C361, C91, C124, C169 and C567.

In one embodiment, each albumin motif contains a total of 2-4 unpaired cysteine residues.

In one aspect, the chimeric molecule is prepared by a process comprising:
a. mutagenizing a nucleic acid sequence by replacing one or more amino acid residues in the albumin motif by cysteine to encode the cysteine engineered fusion molecule;
b. expressing the cysteine engineered fusion molecule; and
c. isolating the cysteine engineered fusion molecule.

In one embodiment, the invention provides an isolated chimeric molecule, which comprises the antibody-albumin fusion molecule, which further comprises at least two antibiotic molecules, wherein the antibiotic molecules are conjugated to the unpaired cysteine residues of the albumin motif, optionally through a linker; wherein the antibody binds to one or more antigens on the surface of a bacterium.

In one embodiment, the antibody of said isolated chimeric molecule binds to an antigen or antigens on a bacterium with multidrug resistance.

In one embodiment, the antibody in the isolated chimeric molecule binds to an antigen or antigens on Methicillin-resistant Staphylococcus aureus (MRSA).

In one embodiment, the antibody in the isolated chimeric molecule binds to an antigen or antigens selected from the group consisting of CifA, ABC Transporter, Lipoteioic Acid, Iron Surface Determinant B, and Poly-N-Acetyl-Glucosamine (PNAG).

In one embodiment, the antibody in the isolated chimeric molecule in the isolated chimeric molecule is selected from the group consisting of F598, Aurexis, Aurograb, and Pagibaximab.

In one embodiment, the antibiotic molecule in the isolated chimeric molecule is selected from the group consisting of daptomycin, Trimethoprim/sulfamethoxazole (TMP/SMX), vancomycin, Linezolid, Quinupristin/dalfopristin, and Ceftarolin.

In one embodiment, the antibody in the chimeric molecule is F598, which comprises light chains with at least 98%, 99% or 100% identity to SEQ ID NO:18 and heavy chains with at least 95%, 96%, 97%, 98%, 99% or 100% identity to SEQ ID NO:19.; and wherein the drug molecule is daptomycin.

In another embodiment, the invention provides an isolated chimeric molecule comprises said antibody-albumin fusion molecule, which further comprises at least two cytotoxic drug molecules, wherein the drug molecules are conjugated to the unpaired cysteine residues of the albumin motif, preferably through a linker.

In one embodiment, each of the chimeric molecules comprises 2-96 chemotherapy agent molecules. In one embodiment, the chimeric molecule comprises 2-96 chemotherapy agent molecules; wherein the chemotherapy molecules are attached to the antibody-albumin fusion molecule non-covalently.

In one embodiment, the antibody binds to one or more antigens on the surface of a cancer cell.

In one embodiment, the antibody binds to one or more antigens on the surface of a cancer cell, wherein the cancer cell has upregulated macropinocytosis.

In one embodiment, the antibody binds to one or more antigens on the surface of a cancer cell, wherein the cancer cell comprises one or more mutations in RAS family genes.

In one embodiment, the RAS mutation is an H-RAS mutation.

In one embodiment, the RAS mutation is a K-RAS mutation.

In one aspect, the invention provides an isolated chimeric molecule, which comprises an antibody, at least one albumin or albumin fragment, and at least one cytotoxic drug molecule, an active peptide, or an antibiotics molecule, wherein the drug molecule is conjugated to said albumin or albumin fragment, optionally through a linker; the albumin in the isolated chimeric molecule is human serum albumin and has at least 90% sequence identity to the amino acid sequence of SEQ ID NO:1.

In one embodiment, the isolated chimeric molecule contains peptide linker or linkers between the albumin motif and the heavy chain or the light chain of the antibody; wherein the peptide linker or linkers contain at least one cysteine residue.

In one embodiment, there is a short peptide of 5 or more amino acids fused to the C-terminal of the albumin motif or motifs; wherein the short peptide contains one cysteine residue.

In one embodiment, the antibody in said chimeric molecule binds to one or more antigens on a cancer cell.

In one embodiment, the cancer cell has upregulated macropinocytosis process.

In one embodiment, the targeted cancer cell comprises one or more mutations in RAS family genes.

In one embodiment, the RAS mutation is an H-RAS mutation. In one embodiment, the RAS mutation is a K-RAS mutation.

In one embodiment, the antibody binds to an antigen selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

In one embodiment, the cytotoxic drug molecule is selected from the group consisting of microtubule disrupting agents, DNA modifying agents, RNA polymerase inhibitors, and topoisomerase I inhibitors.

In one embodiment, the cytotoxic drug molecule is selected from the group consisting of azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, duocarmycin, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, monomethyl auristatin E (MMAE), monomethyl auristatin F (MMAF), phenylbutyrate, prednisone, procarbazine, paclitaxel, pentostatin, pyrrolobenzodiazepine (PBD), semustine, SN-38, streptozocin, tamoxifen, taxanes, taxol, testosterone propionate, thalidomide, thioguanine, thiotepa, teniposide, topotecan, uracil mustard, vinblastine, vinorelbine, and vincristine.

In one embodiment, the cytotoxic drug molecules are individually and covalently linked to the free thiol side chains of the albumins or albumin fragments of the chimeric molecule through a linker or linkers; wherein the linker or linkers contain a thiol reactive group.

In one embodiment, the thiol reactive group is a maleimide group.

In one embodiment, the maleimide group is a N-alkyl maleimide group.

In one embodiment, the succinimide thioethers formed by maleimide-thiol conjugation is hydrolyzed to its ring-opened counterpart.

In one embodiment, the antibody domain of the chimeric molecule binds to one or more antigens on a cancer cell, and wherein the chimeric molecule is internalized upon the binding of the chimeric molecule to the antigen.

In one embodiment, the isolated chimeric molecule comprises 2-40 copies of the albumins or albumin fragments.

In one embodiment, the albumin or albumin fragment is fused to the C-terminus of the heavy chain of antibody, optionally through peptide linkers.

In one embodiment, the isolated chimeric molecule comprises 2-40 copies of the albumin or albumin fragments; wherein 2-40 albumin or albumin fragments are optionally linked to each other in tandem, optionally through peptide linkers.

In one embodiment, the chimeric molecule contains 2, 4, 6, 8, 10, 12, 16, 18 or 20 copies of the albumin or albumin fragments.

In one embodiment, the isolated chimeric molecule comprises 2-160 cytotoxic molecules.

In one embodiment, the antibody of said isolated chimeric molecules is selected from:
a. Trastuzumab or a HER2 antibody, which comprises a light chain having an amino acid sequence with at least 98%, 99% or 100% identity to SEQ ID NO:2 and a heavy chain having an amino acid sequence with at least 95%, 96%, 97%, 98%, 99% or 100% identity to SEQ ID NO:3;
b. Rutuximab or a CD20 antibody, which comprises a light chain having an amino acid sequence with at least 98%, 99% or 100% identity to SEQ ID NO:4 and which comprises a heavy chain having an amino acid sequence with at least 95%, 96%, 97%, 98%, 99% or 100% identity to SEQ ID NO:5;
c. Cetuximab or an EGFR antibody, which comprises light chains with amino acid sequence at least 98%, 99% or 100% identical to SEQ ID NO:6 and heavy chains with amino acid sequence at least 95%, 96%, 97%, 98%, 99% or 100% identical to SEQ ID NO:7;
d. Panitumumab or an EGFR antibody, which comprises light chains with amino acid sequence at least 98%, 99% or 100% identical to SEQ ID NO:8 and heavy chains with amino acid sequence at least 95%, 96%, 97%, 98%, 99% or 100% identical to SEQ ID NO:9;
e. Brentuximab or a CD30 antibody, which comprises a light chain having an amino acid sequence with at least 98%, 99% or 100% identity to SEQ ID NO:10 and which comprises a heavy chain having an amino acid sequence with at least 95%, 96%, 97%, 98%, 99% or 100% identity to SEQ ID NO:11;
f. A DLL-3 antibody which binds to the EGF domain of the DLL-3 molecule;
g. DLL-3 antibody binds to the DSL domain of the DLL-3 molecule;
h. DLL-3 antibodies DL301, DL302, DL305, DL306, DL308, DL309, and DL312, and their humanized versions;
i. A C-MET antibody which comprises a light chain having an amino acid sequence with at least 98%, 99% or 100% identity to SEQ ID NO:12 and which comprises a heavy chain having an amino acid sequence with at least 95%, 96%, 97%, 98%, 99% or 100% identity to SEQ ID NO:13;
j. A GPC-3 antibody which binds to an epitope located after the 374th amino acid of the GPC-3 molecule;
k. A GPC-3 antibody which binds to the heparin sulfate glycan of the GPC-3 molecule;
l. GPC-3 antibody GC33 and its humanized versions;
m. A GPC-3 antibody which comprises a light chain having an amino acid sequence with at least 98%, 99% or 100% identity to SEQ ID NO:14 and which comprises a heavy chain having an amino acid sequence with at least 95%, 96%, 97%, 98%, 99% or 100% identity to SEQ ID NO:15;
n. An EGFR antibody;
o. The EGFR antibody mAb806;
p. A Trop-2 antibody;
q. A trop-2 antibody which comprises the same complementarity determining regions (CDRs) as that of the humanized RS7 antibody, wherein the CDRs of the light chain variable region of the antibody or fragment thereof comprise CDR1 comprising the amino acid sequence of KASQDVSIAVA (SEQ ID NO:49); CDR2 comprising the amino acid sequence of SASYRYT (SEQ ID NO:50); and CDR3 comprising the amino acid sequence of QQHYITPLT (SEQ ID NO:51); and wherein the CDRs of the heavy chain variable region of the antibody or fragment thereof comprise CDR1 comprising the amino acid sequence of NYGMN (SEQ ID NO:46); CDR2 comprising the amino acid sequence of WINTYTGEPTYTDDFKG (SEQ ID NO:47) and CDR3 comprising the amino acid sequence of GGFGSSYWYFDV (SEQ ID NO:48).
r. A mesothelin antibody;
s. A mesothelin-binding scFv or antibody which comprises the same complementarity determining regions (CDRs) as that of SS1, wherein the CDRs of the light chain variable region of the antibody or fragment thereof comprise CDR1 comprising the amino acid sequence of SASSSVSYMH (SEQ ID NO:55); CDR2 comprising the amino acid sequence of DTSKLAS (SEQ ID NO: 56); and CDR3 comprising the amino acid sequence of QQWSGYPLT (SEQ ID NO:57) and wherein the CDRs of the heavy chain variable region of the antibody or fragment thereof comprise CDR1 comprising the amino acid sequence of GYTMN (SEQ ID NO:52); CDR2 comprising the amino acid sequence of LITPYNGASSYNQKFRG (SEQ ID NO:53) and CDR3 comprising the amino acid sequence of GGYDGRGFDY(SEQ ID NO:54).
t. A Claudin 18.2 antibody which does not bind to Claudin 18.1 or binds to Claudin 18.1 with at least 10 times weaker in term of binding affinity;
u. A Claudin 18.2 antibody which comprises a light chain having an amino acid sequence with at least 98%, 99% or 100% identity to SEQ ID NO:16 and which comprises a heavy chain having an amino acid sequence with at least 95%, 96%, 97%, 98%, 99% or 100% identity to SEQ ID NO:17;
v. An antibody which binds to two different epitopes of an antigen selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6; and
w. A bispecific antibody which binds to two antigens selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

In one embodiment, the antibody in the chimeric molecule binds to GPC3, wherein the albumin motif fused to the heavy chain of the antibody, wherein the antibody heavy chain-albumin fusion has an amino acid sequence 99% or 100% identity to SEQ ID NOS: 41 or 42.

In one embodiment, the antibody binds to DLL-3; wherein the cytotoxic agent is an DNA-modifying agent.

In one embodiment, the DNA modifying agent is selected from PBD dimers, Calicheamicins and Duocarymycins.

In one embodiment, the antibody binds to GPC-3, mesothelin, Claudin 18.2, GCC or Trop-2; wherein the cytotoxic agent is a microtubule disrupting agent.

In one embodiment, the microtubule disrupting agent is selected from Auristatins and Maytansines.

In one embodiment, the microtubule disrupting agent is selected from MMAE and MMAF.

In one embodiment, the antibody is the EGFR antibody mAb806; wherein the cytotoxic agent is a microtubule disrupting agent.

In one embodiment, the microtubule disrupting agent is selected from MMAE and MMAF.

In one embodiment, the antibody binds to EGFR, GPC-3, mesothelin, Claudin 18.2 but not to Claudin 18.1 or binding to Claudin 18.1 but with at least 10 times lower affinity, GCC or Trop-2; wherein the cytotoxic agent is a topoisomerase I inhibitor.

In one embodiment, the topoisomerase I inhibitor is SN-38.

In one embodiment, the antibody comprises 2, 4, 6, 8, or 10 human serum albumin molecules or albumin variants; wherein each human serum albumin molecile or albumin variant contains 1, 2, 3 or 4 unpaired cysteine residues; and wherein said isolated chimeric molecule comprises 2 to 40 cytotoxic drug molecules.

In one aspect, the invention comprises an oligomer, a polymer, or nanoparticle, which contains at least one isolated chimeric molecule as described above; and wherein the nanoparticle further contains chemotherapy agents.

In one aspect, the invention comprises a pharmaceutical composition comprising an isolated chimeric molecule and a pharmaceutically acceptable carrier.

In one aspect, the invention comprises a method for treating cancer in a subject, said method comprising administering to a subject in need of such a treatment a pharmaceutical composition as described above.

In one embodiment, the cancer cells contain mutation in RAS family genes. In one embodiment, the RAS mutation is H-RAS. In one embodiment, the RAS mutation is K-RAS.

In one embodiment, the cancer treatment is administered to patients identified positive with both the antigens targeted by the said chimeric molecule, and RAS mutations, as tested by using companion diagnostic biomarker assays suitable for testing the antigen and RAS mutation.

In one embodiment, the cancer is selected from the group consisting of colorectal cancer, stomach or gastric cancer, squamous cell carcinomas, prostate cancer, pancreatic cancer, lung cancer, cholangiocarcinoma, breast cancer and ovarian cancer.

In yet another embodiment, chimeric molecule, which comprises:
a. an antibody;
b. one or more albumin domains; and
c. one or more peptides
wherein the albumin domain is selected from an intact albumin molecule, an albumin fragment, or an albumin variant;
wherein the albumin domain(s) is operationally linked to the antibody molecule, optionally through a linker; and
wherein the peptide(s) is operationally linked to the albumin domain(s), optionally through a linker or linkers.

In one embodiment, the peptide in the chimeric molecule contains at least one unnatural amino acid.

In one embodiment, the peptide in the chimeric molecule is an agonist, which binds to and activate one, two or all three of the following receptors: a) human GLP-1 receptor; b) human Gastric Inhibitory Polypeptide (GIP) receptor, and c) human Glucagon receptor; and wherein there is an optional linker between the peptide agonist and the albumin domain of the chimeric molecule.

In one embodiment, the peptide in the chimeric molecule is selected from the group consisting of 1) GLP-1 and its analogs; 2) exendin-4 and its analogs; 3) GIP and its analogs; and 4) Oxyntomodulin and its analogs.

In one embodiment, the peptide in the chimeric molecule has amino acid sequence selected from the group consisting of SEQ ID NOS: 16-25.

In one embodiment, the antibody in the chimeric molecule binds to human PCSK9, human Glucagon Receptor, and/or human ASGR1.

In one embodiment, the antibody in the chimeric molecule binds to human PCSK9; wherein the albumin molecule is fused to the C-terminals of the heavy chains of the antibody; wherein the heavy chain-albumin fusion has an amino acid sequence with at least 95%, at least 96%, at least 97%, at least 98%, at least 99%, or 100% sequence identity to SEQ ID NO:36; and wherein the chimeric molecule further comprises a light chain with amino acid sequence at least 98%, at least 99%, or 100% sequence identity to SEQ ID NO:34.

In one embodiment, the antibody in the chimeric molecule binds to human Glucagon Receptor; wherein the albumin molecule is fused to the C-terminals of the heavy chains of the antibody; wherein the heavy chain-albumin fusion has an amino acid sequence with at least 95%, at least 96%, at least 97%, at least 98%, at least 99%, or 100% sequence identity to SEQ ID NO:39; and wherein the chimeric molecule further comprises a light chain with amino acid sequence at least 98%, at least 99%, or 100% sequence identity to SEQ ID NO:37.

In one embodiment, the peptide in the chimeric molecule is a PTH/PTHrP peptide comprising a PTH/PTHrP modulating domain.

In one embodiment, the peptide has an amino acid sequence with at least 90%, at least 95%, at least 96%, at least 97%, at least 98%, at least 99%, or 100% sequence identity to SEQ ID NOS: 26 and 27.

In one embodiment, the antibody chimeric molecule binds to a human RANK ligand.

In one embodiment, the antibody is Denosumab.

In one embodiment, the albumin molecule in the chimeric molecule is fused to the C-terminals of the heavy chains of the antibody; wherein the heavy chain-albumin fusion has an amino acid sequence with at least 95%, at least 96%, at least 97%, at least 98%, at least 99%, or 100% sequence identity to SEQ ID NO:33; and wherein the chimeric molecule further comprises a light chain with amino acid sequence at least 98%, at least 99%, or 100% sequence identity to SEQ ID NO:31.

### BRIEF DESCRIPTION OF THE DRAWINGS

FIG. 1: Schematic drawings of novel ADC vs traditional ADC.

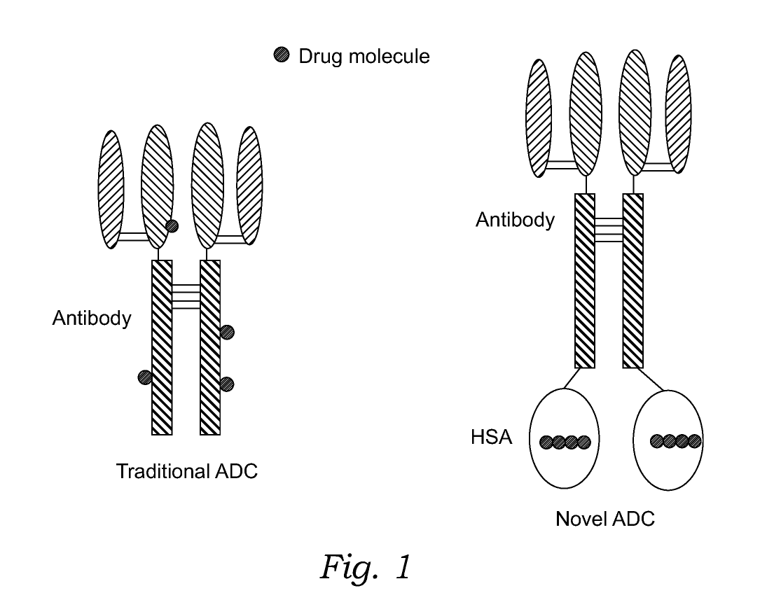

FIG. 2. Purification of Anti-GPC3-AB#1 using Capto Q Impres Chromatography. The column was loaded with a Protein A Affinity Chromatography pool.

FIG. 3. SEC-HPLC purity analysis of the Protein A Affinity Chromatography pool of Anti-GPC3-AB#1.

FIG. 4. SEC-HPLC purity analysis of the Capto Q Impres Chromatography pool of Anti-GPC3-AB#1.

FIG. 5A-F. Expression of GPC3 on HEPG2 and ChoK1/GPC3 transfection cells. Black lines represent Isotype control antibody. Red and green lines represent GPC3 antibody(duplicate).

FIG. 6A-L. Internalization of GPC-3 antibody and chimeric molecules. The antibody and the chimeric molecules were conjugated with pHAb dye. Black lines represent on ice. Red lines represent 37° C. culture.

FIG. 7. Anti-GPC3 Antibody and Chimeric Molecules Internalization in HepG2 Cells.

### DETAILED DESCRIPTION OF THE INVENTION

The present invention provides a chimeric molecule, where the chimeric molecule comprises an albumin motif, an antibody, and at least one, preferably two or more drug molecules. In one aspect, the albumin motif is derived from an albumin variant that is mutated to comprise at least two non-linking cysteine residues, which are each linked to a drug molecule, directly or via a chemical linker. The albumin motif and the antibody can be linked directly, as a single chain molecule, or via a chemical or flexible amino acid linker. The compositions of the invention are used to treat infections caused by drug-resistant bacteria. More importantly, compositions of the invention are used to treat cancer, cancers with enhanced macropinocytosis, and in particular cancers comprising a RAS mutation, such as colorectal cancer, stomach cancer, squamous cell carcinomas, prostate cancer, pancreatic cancer, lung cancer, cholangiocarcinoma, breast cancer and ovarian cancer. In addition, compositions of the invention are also used to treat metabolic diseases such as diabetics as well as bone diseases such as osteoporosis.

The antibody can be an anti-cancer antigen antibody, an anti-cell receptor antibody, an antibody that recognizes an antigen associated with obesity, heart disease, diabetes, or liver disease, an antibody against a multi drug resistant bacterial antigen, an anti-pathogenic antibody, e.g., against a bacterium, a virus such as HCV, HBV, HIV or HPV, a protozoa or a multi-cellular parasite. In one embodiment, the antibody is selected from a single chain variable fragment (scFv), a Fab, a single-head antibody, a single chain antibody, and a monovalent antibody. Conceivably, binding domains other than the antibodies disclosed here, including but not limited to aptamers, high affinity peptides created from combinatory libraries, can also be used to replace the antibody domains.

Though albumin-antibody fusions have been mentioned previously (see, e.g., U.S. Pat. No. 6,905,688), the patent did not disclose any albumin variants with more than one unpaired cysteine residue. The invention in the U.S. Pat. No. 6,905,688 was for the purpose of extending the half-lives of therapeutic proteins and did not disclose any antibody-drug conjugations to the free thiol side chains of the albumin.

The drug can be a chemotherapeutic agent, an antibiotic, or an anti-fungal, parasitic or anti-viral agent (e.g., ribavirin or acyclovir). The peptide can be a cellular receptor agonist or antagonist.

Antibody-drug conjugates (ADC) have been intensively researched as therapeutics to treat cancers. The drug molecules are selected from chemotherapeutic agents, which are often hydrophobic. To this date, the ADC molecules are typically formed by conjugating the drug molecules, through a linker, to amine groups present in the antibody molecule or free thiol groups introduced into the antibody molecule through engineering. The number of drug molecules that are able to be conjugated to a given antibody molecule (the drug/antibody ratio, or DAR) is limited, typically no more than four per antibody, at least in part due to the hydrophobicity of the drug molecules, which would destabilize the antibody molecule if the number of drug molecules per antibody is more than four. There are situations when the density of the target molecules on cancer cell surface are limited, and consequently a higher DAR is required in order to achieve therapeutic efficacy.

Shielded Hydrophobicity, Improved Stability, Prolonged In Vivo Half-Life, and Enhanced Therapeutic Index:

Most of the ADC molecules in clinical trials have half-lives of about 7 days or shorter (Antoine Deslandes, mAbs 6:4, 859-870, 2014), which were in general significantly shorter than the naked antibodies. While the loss of the drugs from antibody-drug conjugates over time contributed significantly to the reduced half-lives, the hydrophobicity of the conjugates because of the drug molecules may have also contributed significantly to the reduction of half-lives in vivo (Lyon et al., “Reducing hydrophobicity of homogeneous antibody-drug conjugates improves pharmacokinetics and therapeutic index”. Nature Biotechnology 33, 733-735 (2015)). It was reported that “hydrophobicity and plasma clearance also correlated with the hepatic uptake of these ADCs” (Lyon et al., 2015). In addition, the increased exposure also translated into higher therapeutic efficacy as demonstrated in the xenograft animal model (Lyon et al., 2015).

ADC created with advanced linker chemistry had significantly higher stability and lower premature release of the payloads in plasma. Maleimides are commonly used to attached the drug molecules to the thiol groups of proteins and antibodies. The formed conjugates may be instable and cleaved in vivo due to reactions such as thiol exchange. Conjugates made with electron-withdrawing maleimides can purposefully hydrolyzed to their ring-opened counterparts in vitro to ensure in vivo stability (Fontaine et al., Bioconjug Chem. 2015 Jan. 21; 26(1):145-52). The cysteine-linked antibody drug conjugates may also be stabilized using N-aryl maleimids (Christie et al., J Control Release 2015 Dec. 28; 220(Pt B):660-70).

One of the novel approach of resolving the hydrophobicity issue of the current ADC technology is to conjugating the drugs to the albumin motifs of antibody-albumin fusion molecules (see, e.g., Provisional Patent Application No. 62/369,649, herein incorporated by reference in its entirety). Due to the unique characteristics of the albumin molecule, conjugation of hydrophobic drug molecules to the albumin motifs would shield the hydrophobicity of the drug molecules. This approach reduced the overall hydrophobicity of the ADC molecules. Together with advanced linker chemistry, ADC molecules created with this novel technology can result into significantly enhanced stability and reduced propensity for aggregation, reduced immunogenicity, and much longer half-lives in vivo than conventional ADCs and exposure. Consequently, this invention can yield ADC molecules with significantly improved therapeutic index through longer exposure, higher efficacy and lower toxicity.

Enhanced Drug-Antibody Ratios:

Lyon et al. (2015) reported that higher drug/antibody ratio (DAR) correlated to higher anticancer activity in mouse model, provided that hydrophobicity was appropriately managed. Conjugation of drugs to the albumin motifs in the antibody-albumin fusion molecules also allows higher drug/antibody ratio (DAR). Higher DAR may be achieved by fusing a larger number of albumin motifs to the antibody molecules, e.g. 4, 6, 8, 10, or 16 albumins per antibody molecules would allow a DAR of 8, 12, 16, 20, or 32.

Since each albumin molecule may be able to carry up to 4-6 hydrophobic molecules non-covalently, drug molecules may be made dimers, trimers or tetramers and then be conjugated to the free thiol groups of the albumin motifs, which would further enhance the DAR. In addition, linkers can be engineered to link 2 drug war heads per linker.

Alternatively, point mutations may be introduced to the albumin motifs to have 2, 3 or 4 free thiols per albumin motif. For examples, Ser and/or Lys residues at selected sites in the albumin motifs may be mutated to Cys in order to introduce additional free thiol groups.

In summary, combination of fusing multiple albumin motifs per antibody, using drug oligomers, linking two war heads per linker, and/or introduction of additional free thiols into the albumin motifs would allow significantly higher DAR without significantly increasing the exposed hydrophobicity of the ADC molecules.

Macropinocytosis, RAS Mutation Biomarkers and Enhanced Delivery of ADC into Tumor Cells with RAS Mutations:

Cancer cells may have upregulated marcropinocytosis in order to meet the nutrient requirements for their fast growth. Stimulation of EGFR and oncogentic RAS expression can actively induce macropinocytosis in cancer cells (Narkase et al., Sci Rep. 2015; 5: 10300.). Podocytes and colorectal cancer cells activate macropinocytosis through interactions between albumin-associated free fatty acids (FFAs) and GPCRs (Wu et al., Oncogene 32, 5541-5550, 2013; Chung et al., J. Clin. Invest. 125, 2307-2316, 2015).

The mammalian ras gene family comprises H-ras, K-ras, N-ras, encoding H-ras, K-ras, N-ras proteins, respectively, with a similar structure and function. The Ras protein is located in the inner region of the cell membrane, tranforms signals from EGFR to mitogen-activated protein kinases (MAPKs), to control cell growth, proliferation, and motility, as well as metastasis and angiogenesis (Kiaris and Spandidos. Mutations of ras genes in human tumours. International Journal of Oncology. 1995; 7:413-429). The K-ras gene usually contains point mutations at codons 12, 13 and 61, and these mutations often activate the K-ras oncogene (Schubbert et al., Hyperactive Ras in developmental disorders and cancer. Nat Rev Cancer. 2007; 7:295-308; Bos et al., Prevalence of ras gene mutations in human colorectal cancers. Nature. 1987; 327:293-297). The K-ras mutation status is associated with the therapeutic efficacy of EGFR-targeting monoclonal antibodies, rendering patients with K-ras mutation as not suitable for Erbitux treatment (Benvenuti et al., Oncogenic activation of the RAS/RAF signaling pathway impairs the response of metastatic colorectal cancers to anti-epidermal growth factor receptor antibody therapies. Cancer Res. 2007; 67:2643-2648).

More than 30% of all human cancers—including 95% of pancreatic cancers and 45% of colorectal cancers—are driven by mutations of the RAS family of genes. Among other changes, oncogenic Ras promotes glucose fermentation and glutamine use to supply central carbon metabolism (White, Genes & Dev. 2013. 27:2065-2071). It was also demonstrated by Commisso et al. (Nature 497, 633-637, 2013) that oncogenic RAS protein was able to stimulating the macro-pinocytosis of the cancer cells, a process the cancer cells utilized to obtain nutrients for rapid growth. As the most abundant plasma protein in the blood, albumin is the desired cargo for the macro-pinocytosis process.

Conjugation of anticancer drugs to albumin motifs fused to antibodies which bind targets on cancer cells with enhanced macropinocytosis, specifically the ones with RAS oncogenes will lead to double selectivity in the delivery of cancer drugs: first through the antigen targets, and then through the macro-pinocytosis process. A typical ADC relies on antibody-target internalization to deliver the anticancer drugs inside the cancer cells. In this case, the separate macropinocytosis also aids in the internalization of the albumin-based ADC molecules. Antibodies which are not internalized may also be utilized to make ADC in this case. In addition, macropinocytosis also allow cancer cells to take in large vehicles such as nanoparticles, liposomes, large molecules such as antibody-albumin-drug conjugates (AADC), and oligomers and polymers of AADC.

Because more than 30% of all cancer cells contain RAS mutations, many of the antigens on cancer cell surface, such as the ones shown in Table 2 above, can be targeted by this disclosed technology.

Since RAS mutations have been found in 95% of pancreatic cancers and 45% of colorectal cancers, antigens on those two cancer cells are among the most suitable targets to design ADC based on this disclosed technology.

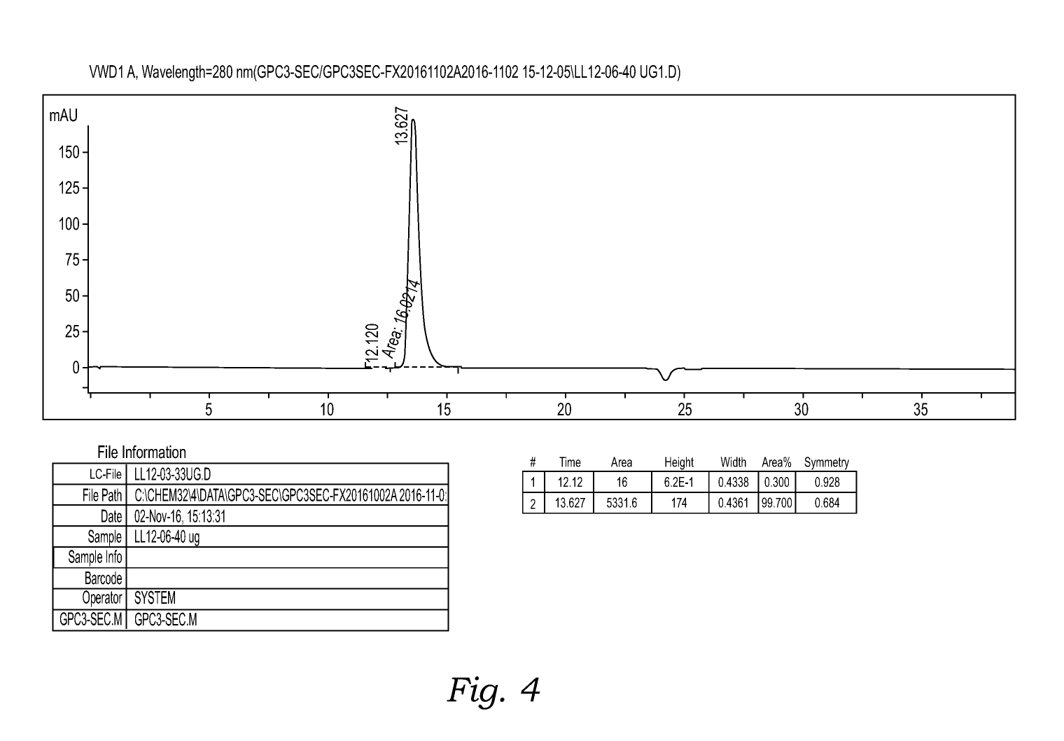

In one aspect, the albumin motif is derived from an albumin variant that is mutated to comprise at least two unpaired cysteine residues. Specifically, the human serum albumin with sequence such as SEQ ID NO:1 comprises one or more of:
(a) substitution of a non-cysteine amino acid with a cysteine at a position corresponding to a position equivalent to any of residues L585, D1, A2, D562, A364, A504, E505, T79, E86, D129, D549, A581, D121, E82, S270, Q397 and A578 of SEQ ID NO:1;
(b) insertion of a cysteine at a position adjacent the N- or C-side of an amino acid which may or may not correspond to a position equivalent to any of residues L585, D1, A2, D562, A364, A504, E505, T79, E86, D129, D549, A581, D121, E82, S270, Q397 and A578 of SEQ ID NO:1;
(c) a cysteine with a free thiol group at a position which may or may not correspond to any of C369, C361, C91, C177, C567, C316, C75, C169, C124 and C558 which may or may not be generated by deletion or substitution of C360, C316, C75, C168, C558, C361, C91, C124, C169 and/or C567; and/or
(d) addition of a cysteine to the N-side of the N-terminal residue of an albumin sequence and/or to the C-side of the C-terminal residue of an albumin sequence such that the net result of the substitution, deletion, addition or insertion events of (a), (b), (c) and (d) is that the number of conjugation competent cysteine residues of the polypeptide sequence is increased relative to the polypeptide prior to the substitution, insertion, deletion and addition events.
Within (a) to (d), above, the residues all of the residues are preferred. However, within each of (a), (b), (c) and (d), the residues are listed in order of decreasing preference. A thio-albumin may or may not include a polypeptide where one or more naturally occurring free-thiol group(s), such as cysteine-34 in HSA (SEQ ID NO:1), is modified to an amino acid which is not cysteine. For example, cysteine may or may not be replaced by an amino acid which has a relatively high conservation score (e.g. 1, 2 or 3 as calculated according to FIG. 4) such as alanine or serine. A thio-album in may or may not include a poly-peptide where one or more naturally occurring free-thiol group(s), such as cysteine-34 in HSA (SEQ ID NO:1) are present.

Additional mutations may also be introduced. For examples, one or more serine residues at the turns between helixes may be substituted with cysteine residue. One or more Lys residues, such as Lys 500, Lys 573, or Lys 574 may also be substituted with cysteine residue.

The albumin motif may be the entire albumin protein (see, e.g., mutated SEQ ID NO:1 as described above), a domain thereof, e.g., the Sudlow I and/or the Sudlow II domain, or a fragment of the protein or domain thereof, e.g., a fragment of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, or 580 amino acids in length. One or more albumin motifs may be linked to the antibody.

The antibody can be targeted to a hematopoietic differentiation antigen, a glycoprotein expressed by solid tumors, a glycolipid, a carbohydrate, a targets of anti-angiogenic mAbs, a growth and differentiation signaling molecule or a stromal and extracellular matrix antigen a shown in Table 1. In one embodiment, the antibody binds to Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6. In one embodiment, the antibody is bispecific and binds to two different antigens. In one embodiment, the antibody binds targets on cancer cells expressing the RAS oncogene, including targets such as GGC, mesothelin, Her3, IGFR1, Trop-2, carbohydrate antigen 19-9, CEA, EpCam, EGFR, and gpA33.

In one embodiment, the antibody comprises a heavy chain and a light chain. In one embodiment, the antibody is selected from a single chain variable fragment (scFv), a Fab, a single-head antibody, a single chain antibody, and a monovalent antibody.

The albumin motif is linked to the antibody directly, via a flexible amino acid linker or optionally by a chemical linker. 1, 2, 3, 4, 5, 6, 7, 8, 9, 10, 11, 12, 13, 14, 15, 16, 17, 18, 19, 20, 22, 24, 26, 28, 30, 32, 34, 36, 38, 40 or more albumin motifs can be linked to the antibody. In one embodiment, the albumin motif is fused to the C-terminals of the heavy chains of the antibody.

At least one, or at least two or more of the same or different cytotoxic drugs are linked to the albumin motif via a cysteine residue. In one embodiment, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160 or more cytotoxic drug molecules are linked to the albumin motif.

The cytotoxic drug molecule can be selected from the group consisting of microtubule disrupting agents, DNA modifying agents, RNA polymerase inhibitors, and topoisomerase I inhibitors.

The cytotoxic drug can be a chemotherapeutic agent such as azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, duocarmycin, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, monomethyl auristatin E (MMAE), MMAF, phenylbutyrate, prednisone, procarbazine, paclitaxel, pentostatin, pyrrolobenzodiazepine (PBD), semustine, SN-38, streptozocin, tamoxifen, taxanes, taxol, testosterone propionate, thalidomide, thioguanine, thiotepa, teniposide, topotecan, uracil mustard, vinblastine, vinorelbine, and vincristine.

In general, the drug molecules attached to the linkers, which further conjugate to free amine or free thiol groups on the antibody of the ADC.

Anticancer nanoparticles are developed to capsuling hydrophobic drug molecules. Among them are albumin-based nanoparticles (e.g., albumin bound paclitaxel (ABRAXANE©) and liposome-based nanoparticles. For example, compositions and methods of making albumin-based nanoparticles have been disclosed in U.S. Pat. No. 8,846,771. Binding domains such as antibodies, which binds to targets on cancer cell surfaces, can be introduced to the albumin-based and liposome-based nanoparticles. Specifically, antibody-albumin fusion molecules can be introduced into the nanoparticles during the production process, wherein the nanoparticles also contain cytotoxic agents.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The present composition encompasses amino acid substitutions in proteins and peptides, which do not generally alter the activity of the proteins or peptides (H. Neurath, R. L. Hill, The Proteins, Academic Press, New York, 1979). In one embodiment, these substitutions are “conservative” amino acid substitutions. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Analogue as used herein denotes a peptide, polypeptide, or protein sequence which differs from a reference peptide, polypeptide, or protein sequence. Such differences may be the addition, deletion, or substitution of amino acids, phosphorylation, sulfation, acrylation, glycosylation, methylation, farnesylation, acetylation, amidation, and the like, the use of non-natural amino acid structures, or other such modifications as known in the art.

The term “unnatural amino acids” as used herein refers to amino acids other than the 20 typical amino acids found in the proteins in our human body. Unnatural amino acids are non-proteinogenic amino acids that either occur naturally or are chemically synthesized. They may include but are not limited to aminoisobutyric acid (Aib), β-amino acids (β³ and β²), homo-amino acids, proline and pyruvic acid derivatives, 3-substituted alanine derivatives. Glycine derivatives, ring-substituted phenylalanine and tyrosine derivatives, Linear core amino acids, diamino acids, D-amino acids and N-methyl amino acids.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into a Fab′ monomer. The Fab′ monomer is essentially Fab with part of the hinge region (see Fundamental Immunology, Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Accordingly, in either aspect of the invention, the term antibody also embraces minibodies, diabodies, triabodies and the like. Diabodies are small bivalent biospecific antibody fragments with high avidity and specificity. Their high signal to noise ratio is typically better due to a better specificity and fast blood clearance increasing their potential for diagnostic and therapeutic targeting of specific antigen (Sundaresan et al., J Nucl Med 44:1962-9 (2003). In addition, these antibodies are advantageous because they can be engineered if necessary as different types of antibody fragments ranging from a small single chain Fv to an intact IgG with varying isoforms (Wu & Senter, Nat. Biotechnol. 23:1137-1146 (2005)). In some embodiments, the antibody fragment is part of a diabody. In some embodiments, in either aspect, the invention provides high avidity antibodies for use according to the invention.

In some embodiments, CDR regions may be defined using the Kabat definition, the Chothia definition, the AbM definition, the contact definition, or any other suitable CDR numbering system.

Diabodies, first described by Hollinger et al., PNAS (USA) 90(14): 6444-6448 (1993), may be constructed using heavy and light chains disclosed herein, as well as by using individual CDR regions disclosed herein. Typically, diabody fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VH and VL domains of another fragment, thereby forming two antigen-binding sites. Triabodies can be similarly constructed with three antigen-binding sites. An Fv fragment contains a complete antigen-binding site which includes a VL domain and a VH domain held together by non-covalent interactions. Fv fragments embraced by the present invention also include constructs in which the VH and VL domains are crosslinked through glutaraldehyde, intermolecular disulfides, or other linkers. The variable domains of the heavy and light chains can be fused together to form a single chain variable fragment (scFv), which retains the original specificity of the parent immunoglobulin. Single chain Fv (scFv) dimers, first described by Gruber et al., J. Immunol. 152(12):5368-74 (1994), may be constructed using heavy and light chains disclosed herein, as well as by using individual CDR regions disclosed herein. Many techniques known in the art can be used to prepare the specific binding constructs of the present invention (see, U.S. Patent Application Publication No. 2007/0196274 and U.S. Patent Application Publication No. 2005/0163782, which are each herein incorporated by reference in their entireties for all purposes, particularly with respect to minibody and diabody design).

Bispecific antibodies can be generated by chemical cross-linking or by the hybrid hybridoma technology. Alternatively, bispecific antibody molecules can be produced by recombinant techniques. Dimerization can be promoted by reducing the length of the linker joining the VH and the VL domain from about 15 amino acids, routinely used to produce scFv fragments, to about 5 amino acids. These linkers favor intrachain assembly of the VH and VL domains. Any suitable short linker can be used. Thus, two fragments assemble into a dimeric molecule. Further reduction of the linker length to 0-2 amino acids can generate trimeric (triabodies) or tetrameric (tetrabodies) molecules.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., in Monoclonal Antibodies and Cancer Therapy, Alan R. Liss, Inc., pp. 77-96 (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; and WO 92/200373).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity, neurodegeneration or pathological inflammation, normal human cells or tissues.

An “immunoregulator” refers to a substance, an agent, a signaling pathway or a component thereof that regulates an immune response. “Regulating,” “modifying” or “modulating” an immune response refers to any alteration in a cell of the immune system or in the activity of such cell. Such regulation includes stimulation or suppression of the immune system which may be manifested by an increase or decrease in the number of various cell types, an increase or decrease in the activity of these cells, or any other changes which can occur within the immune system. Both inhibitory and stimulatory immunoregulators have been identified, some of which may have enhanced function in the autoimmune microenvironment.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

Construction of suitable vectors containing the desired sequences and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

Nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273(52):35095-35101 (1998).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The contacting of the patient with the antibody-albumin-drug conjugates, can be by administering the conjugates to the patient intravenously, intraperitoneally, intramuscularly, intratumorally, or intradermally. In some embodiments the antibody-album in-drug conjugates is co-administered with an additional immunotherapy agent.

The term “recombinant” as used herein refers to a polypeptide produced through a biological host, selected from a mammalian expression system, an insect cell expression system, a yeast expression system, and a bacterial expression system.

The term “formulation” as used herein refers to the antibodies disclosed herein and excipients combined together which can be administered and has the ability to bind to the corresponding receptors and initiate a signal transduction pathway resulting in the desired activity. The formulation can optionally comprise other agents.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the antibody disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and preferably is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose, or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the peptide is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

In still another aspect, the present specification provides a method for preventing or treating a disease comprising the step of administering to a subject the chimeric protein or the pharmaceutical composition including the same.

“Cancer” refers to human cancers and carcinomas, sarcomas, adenocarcinomas, etc., including leukemias, lymphomas, solid tumors, kidney, breast, lung, kidney, bladder, urinary tract, urethra, penis, vulva, vagina, cervical, colorectal, ovarian, prostate, pancreas, stomach, brain, head and neck, skin, uterine, testicular, esophagus, liver cancer, and squamous cell carcinomas, and cholangiocarcinoma.

“Nanoparticle” refers materials with overall dimensions in the nanoscale, i.e., under 200 nm. In recent years, these materials have emerged as important players in modern medicine, with clinical applications ranging from contrast agents in imaging to carriers for drug and gene delivery into tumors, e.g., liposomes, polymers, dendrimers, etc. (see, e.g., Murthy, Int J Nanomedicine (2007) 2(2):129-141).

As used herein, the term “prevention” means all of the actions by which the occurrence of the disease is restrained or retarded.

As used herein, the term “treatment” means all of the actions by which the symptoms of the disease have been alleviated, improved or ameliorated. In the present specification, “treatment” means that the symptoms of a disease are alleviated, improved or ameliorated by administration of the chimeric molecules disclosed herein.

As used herein, the term “administration” means introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

In the present specification, the term “subject” is those suspected of having or diagnosed with a disease. However, any subject to be treated with the pharmaceutical composition disclosed herein is included without limitation. The pharmaceutical composition including the chimeric molecule disclosed herein is administered to a subject suspected of having a disease as disclosed herein.

The therapeutic method of the present specification may include the step of administering the composition including the antibody at a pharmaceutically effective amount. The total daily dose should be determined through appropriate medical judgment by a physician, and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In still another aspect, the present specification provides a use of the therapeutic protein or the pharmaceutical composition including the same in the preparation of drugs for the prevention or treatment of a disease.

In one embodiment, the dose of the composition may be administered daily, semi-weekly, weekly, bi-weekly, tri-weekly, or monthly. The period of treatment may be for a week, two weeks, a month, two months, four months, six months, eight months, a year, or longer. The initial dose may be larger than a sustaining dose. In one embodiment, the dose ranges from a tri-weekly dose of at least 0.01 mg, at least 0.25 mg, at least 0.3 mg, at least 0.5 mg, at least 0.75 mg, at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 50 mg, at least 55 mg, at least 60 mg, at least 65 mg, at least 70 mg, at least 100 mg, at least 200 mg, at least 300 mg, at least 500 mg, or at least 1000 mg. In one embodiment, a tri-weekly dose may be at most 0.5 mg, at most 0.75 mg, at most 1 mg, at most 1.25 mg, at most 1.5 mg, at most 2 mg, at most 2.5 mg, at most 3 mg, at most 4 mg, at most 5 mg, at most 6 mg, at most 7 mg, at most 8 mg, at most 9 mg, at most 10 mg, at most 15 mg, at most 20 mg, at most 25 mg, at most 30 mg, at most 35 mg, at most 40 mg, at most 50 mg, at most 55 mg, at most 60 mg, at most 65 mg, at most 70 mg, at most 100 mg, at most 200 mg, at most 300 mg, at most 500 mg, or at most 1000 mg. In a particular aspect, the tri-weekly dose may range from 0.25 mg to 900 mg, from 1 mg to 200 mg. In an alternative aspect, the weekly dose may range from 10 mg to 900 mg.

TABLE 3
Sequences

| SEQ | | |
|---|---|---|
| ID | | |
| NO | Name | Amino Acid Sequence |
| 1 | Human Serum | DAHKSEVAHRFKDLGEENFKALVLIAFAQYLQQCPFEDHVKLVNE |
| | Albumin (HSA) | VTEFAKTCVADESAENCDKSLHTLFGDKLCTVATLRETYGEMAD |
| | | CCAKQEPERNECFLQHKDDNPNLPRLVRPEVDVMCTAFHDNEE |
| | | TFLKKYLYEIARRHPYFYAPELLFFAKRYKAAFTECCQAADKAACL |
| | | LPKLDELRDEGKASSAKQRLKCASLQKFGERAFKAWAVARLSQR |
| | | FPKAEFAEVSKLVTDLTKVHTECCHGDLLECADDRADLAKYICEN |
| | | QDSISSKLKECCEKPLLEKSHCIAEVENDEMPADLPSLAADFVESK |
| | | DVCKNYAEAKDVFLGMFLYEYARRHPDYSVVLLLRLAKTYETTLE |
| | | KCCAAADPHECYAKVFDEFKPLVEEPQNLIKQNCELFEQLGEYKF |
| | | QNALLVRYTKKVPQVSTPTLVEVSRNLGKVGSKCCKHPEAKRMP |
| | | CAEDYLSVVLNQLCVLHEKTPVSDRVTKCCTESLVNRRPCFSALE |
| | | VDETYVPKEFNAETFTFHADICTLSEKERQIKKQTALVELVKHKPK |
| | | ATKEQLKAVMDDFAAFVEKCCKADDKETCFAEEGKKLVAASQAA |
| | | LGL |
| 2 | Trastuzumab | DIQMTQSPSSLSASVGDRVTITCRASQDVNTAVAWYQQKPGKAP |
| | LC | KLLIYSASFLYSGVPSRFSGSRSGTDFTLTISSLQPEDFATYYCQQ |
| | | HYTTPPTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 3 | Trastuzumab | EVQLVESGGGLVQPGGSLRLSCAASGFNIKDTYIHWVRQAPGKG |
| | HC | LEWVARIYPTNGYTRYADSVKGRFTISADTSKNTAYLQMNSLRAE |
| | | DTAVYYCSRWGGDGFYAMDYWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPP |
| | | KSCDKTHTCPPCPAPELLGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPGK |
| 4 | Trastuzumab | EVQLVESGGGLVQPGGSLRLSCAASGFNIKDTYIHWVRQAPGKG |
| | HC-HSA | LEWVARIYPTNGYTRYADSVKGRFTISADTSKNTAYLQMNSLRAE |
| | | DTAVYYCSRWGGDGFYAMDYWGQGTLVTVSSASTKGPSVFPLA |
| | | PSSKSTSGGTAALGCLVKDYFPEPVTVSWNSGALTSGVHTFPAV |
| | | LQSSGLYSLSSVVTVPSSSLGTQTYICNVNHKPSNTKVDKKVEPP |
| | | KSCDKTHTCPPCPAPELLGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPGK(GGGGS)ₙDAHKSEVAHRFKDLGEENFKALVLIAFAQ |
| | | YLQQCPFEDHVKLVNEVTEFAKTCVADESAENCDKSLHTLFGDKL |
| | | CTVATLRETYGEMADCCAKQEPERNECFLQHKDDNPNLPRLVRP |
| | | EVDVMCTAFHDNEETFLKKYLYEIARRHPYFYAPELLFFAKRYKA |
| | | AFTECCQAADKAACLLPKLDELRDEGKASSAKQRLKCASLQKFG |
| | | ERAFKAWAVARLSQRFPKAEFAEVSKLVTDLTKVHTECCHGDLL |
| | | ECADDRADLAKYICENQDSISSKLKECCEKPLLEKSHCIAEVENDE |
| | | MPADLPSLAADFVESKDVCKNYAEAKDVFLGMFLYEYARRHPDY |
| | | SVVLLLRLAKTYETTLEKCCAAADPHECYAKVFDEFKPLVEEPQN |
| | | LIKQNCELFEQLGEYKFQNALLVRYTKKVPQVSTPTLVEVSRNLG |
| | | KVGSKCCKHPEAKRMPCAEDYLSVVLNQLCVLHEKTPVSDRVTK |
| | | CCTESLVNRRPCFSALEVDETYVPKEFNAETFTFHADICTLSEKE |
| | | RQIKKQTALVELVKHKPKATKEQLKAVMDDFAAFVEKCCKADDKE |
| | | TCFAEEGKKLVAASQAALGL; wherein n = 0, 1, 2, 3, or 4 |
| 5 | Rutuximab LC | QIVLSQSPAILSASPGEKVTMTCRASSSVSYIHWFQQKPGSSPKP |
| | | WIYATSNLASGVPVRFSGSGSGTSYSLTISRVEAEDAATYYCQQ |
| | | WTSNPPTFGGGTKLEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 6 | Rutuximab HC | QVQLQQPGAELVKPGASVKMSCKASGYTFTSYNMHWVKQTPGR |
| | | GLEWIGAIYPGNGDTSYNQKFKGKATLTADKSSSTAYMQLSSLTS |
| | | EDSAVYYCARSTYYGGDWYFNVWGAGTTVTVSAASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKAE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVVS |
| | | VLTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 7 | Rutuximab | QVQLQQPGAELVKPGASVKMSCKASGYTFTSYNMHWVKQTPGR |
| | HC-HSA | GLEWIGAIYPGNGDTSYNQKFKGKATLTADKSSSTAYMQLSSLTS |
| | | EDSAVYYCARSTYYGGDWYFNVWGAGTTVTVSAASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKKAE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVVS |
| | | VLTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK(GGGGS)ₙDAHKSEVAHRFKDLGEENFKALVLIAFA |
| | | QYLQQCPFEDHVKLVNEVTEFAKTCVADESAENCDKSLHTLFGD |
| | | KLCTVATLRETYGEMADCCAKQEPERNECFLQHKDDNPNLPRLV |
| | | RPEVDVMCTAFHDNEETFLKKYLYEIARRHPYFYAPELLFFAKRY |
| | | KAAFTECCQAADKAACLLPKLDELRDEGKASSAKQRLKCASLQK |
| | | FGERAFKAWAVARLSQRFPKAEFAEVSKLVTDLTKVHTECCHGD |
| | | LLECADDRADLAKYICENQDSISSKLKECCEKPLLEKSHCIAEVEN |
| | | DEMPADLPSLAADFVESKDVCKNYAEAKDVFLGMFLYEYARRHP |
| | | DYSVVLLLRLAKTYETTLEKCCAAADPHECYAKVFDEFKPLVEEP |
| | | QNLIKQNCELFEQLGEYKFQNALLVRYTKKVPQVSTPTLVEVSRN |
| | | LGKVGSKCCKHPEAKRMPCAEDYLSVVLNQLCVLHEKTPVSDRV |
| | | TKCCTESLVNRRPCFSALEVDETYVPKEFNAETFTFHADICTLSEK |
| | | ERQIKKQTALVELVKHKPKATKEQLKAVMDDFAAFVEKCCKADDK |
| | | ETCFAEEGKKLVAASQAALGL; wherein n = 0, 1, 2, 3, or 4 |
| 8 | Cetuximab LC | DILLTQSPVILSVSPGERVSFSCRASQSIGTNIHWYQQRTNGSPRL |
| | | LIKYASESISGIPSRFSGSGSGTDFTLSINSVESEDIADYYCQQNN |
| | | NWPTTFGAGTKLELKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| 9 | Cetuximab HC | QVQLKQSGPGLVQPSQSLSITCTVSGFSLTNYGVHWVRQSPGK |
| | | GLEWLGVIWSGGNTDYNTPFTSRLSINKDNSKSQVFFKMNSLQS |
| | | NDTAIYYCARALTYYDYEFAYWGQGTLVTVSAASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPELLGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALPAPIEKTISKAKGQPREPQVYTLPP |
| | | SRDELTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SLSPGK |
| 10 | Panitumumab | DIQMTQSPSSLSASVGDRVTITCQASQDISNYLNWYQQKPGKAP |
| | LC | KLLIYDASNLETGVPSRFSGSGSGTDFTFTISSLQPEDIATYFCQH |
| | | FDHLPLAFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 11 | Panitumumab | QVQLQESGPGLVKPSETLSLTCTVSGGSVSSGDYYWTWIRQSP |
| | HC | GKGLEWIGHIYYSGNTNYNPSLKSRLTISIDTSKTQFSLKLSSVTAA |
| | | DTAIYYCVRDRVTGAFDIWGQGTMVTVSSASTKGPSVFPLAPCS |
| | | RSTSESTAALGCLVKDYFPEPVTVSWNSGALTSGVHTFPAVLQS |
| | | SGLYSLSSVVTVPSSNFGTQTYTCNVDHKPSNTKVDKTVERKCC |
| | | VECPPCPAPPVAGPSVFLFPPKPKDTLMISRTPEVTCVVVDVSHE |
| | | DPEVQFNWYVDGVEVHNAKTKPREEQFNSTFRVVSVLTVVHQD |
| | | WLNGKEYKCKVSNKGLPAPIEKTISKTKGQPREPQVYTLPPSREE |
| | | MTKNQVSLTCLVKGFYPSDIAVEWESNGQPENNYKTTPPMLDSD |
| | | GSFFLYSKLTVDKSRWQQGNVFSCSVMHEALHNHYTQKSLSLSP |
| | | GK |
| 12 | Panitumumab | QVQLQESGPGLVKPSETLSLTCTVSGGSVSSGDYYWTWIRQSP |
| | HC-HSA | GKGLEWIGHIYYSGNTNYNPSLKSRLTISIDTSKTQFSLKLSSVTAA |
| | | DTAIYYCVRDRVTGAFDIWGQGTMVTVSSASTKGPSVFPLAPCS |
| | | RSTSESTAALGCLVKDYFPEPVTVSWNSGALTSGVHTFPAVLQS |
| | | SGLYSLSSVVTVPSSNFGTQTYTCNVDHKPSNTKVDKTVERKCC |
| | | VECPPCPAPPVAGPSVFLFPPKPKDTLMISRTPEVTCVVVDVSHE |
| | | DPEVQFNWYVDGVEVHNAKTKPREEQFNSTFRVSVLTVVHQDW |
| | | LNGKEYKCKVSNKGLPAPIEKTISKTKGQPREPQVYTLPPSREEM |
| | | TKNQVSLTCLVKGFYPSDIAVEWESNGQPENNYKTTPPMLDSDG |
| | | SFFLYSKLTVDKSRWQQGNVFSCSVMHEALHNHYTQKSLSLSPG |
| | | K(GGGGS)ₙDAHKSEVAHRFKDLGEENFKALVLIAFAQYLQQCPFE |
| | | DHVKLVNEVTEFAKTCVADESAENCDKSLHTLFGDKLCTVATLRE |
| | | TYGEMADCCAKQEPERNECFLQHKDDNPNLPRLVRPEVDVMCT |
| | | AFHDNEETFLKKYLYEIARRHPYFYAPELLFFAKRYKAAFTECCQA |
| | | ADKAACLLPKLDELRDEGKASSAKQRLKCASLQKFGERAFKAWA |
| | | VARLSQRFPKAEFAEVSKLVTDLTKVHTECCHGDLLECADDRADL |
| | | AKYICENQDSISSKLKECCEKPLLEKSHCIAEVENDEMPADLPSLA |
| | | ADFVESKDVCKNYAEAKDVFLGMFLYEYARRHPDYSVVLLLRLAK |
| | | TYETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTALV |
| | | ELVKHKPKATKEQLKAVMDDFAAFVEKCCKADDKETCFAEEGKK |
| | | LVAASQAALGL; wherein n = 0, 1, 2, 3, or 4 |
| 13 | Brentuximab | DIVLTQSPASLAVSLGQRATISCKASQSVDFDGDSYMNWYQQKP |
| | (XCD30) LC | GQPPKVLIYAASNLESGIPARFSGSGSGTDFTLNIHPVEEEDAATY |
| | | YCQQSNEDPVVTFGGGTKLEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| 14 | Brentuximab | QIQLQQSGPEVVKPGASVKISCKASGYTFTDYYITWVKQKPGQGL |
| | (XCD30) HC | EWIGWIYPGSGNTKYNEKFKGKATLTVDTSSSTAFMQLSSLTSED |
| | | TAVYFCANYGNYWFAYWGQGTQVTVSAASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSRD |
| | | ELTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | G |
| 15 | Brentuximab | QIQLQQSGPEVVKPGASVKISCKASGYTFTDYYITWVKQKPGQGL |
| | (XCD30) HC- | EWIGWIYPGSGNTKYNEKFKGKATLTVDTSSSTAFMQLSSLTSED |
| | HSA | TAVYFCANYGNYWFAYWGQGTQVTVSAASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSRD |
| | | ELTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | G |
| 16 | Human GLP-1 | HGEGTFTSDVSSYLEEQAAKEFIAWLVKGGG |
| | Analog | |
| 17 | Exendin 4 | HGEGTFTSDLSKQMEEEAVRLFIEWLKNGGPSSGAPPPS |
| 18 | Human | HSQGTFTSDYSKYLDSRRAQDFVQWLMNT |
| | Glucagon | |
| 19 | Human GIP | YAEGTFISDYSIAMDKIHQQDFVNWLLAQKGKKNDWKHNITQ |
| 20 | Human | HSQGTFTSDYSKYLDSRRAQDFVQWLMNTKRNRNNIA |
| | Oxyntomodulin | |
| 21 | Oxyntomodulin | HAibQGTFTSDYSKYLD E KRA K EFVQWLMNT; wherein amino |
| | Analog 1 | acids in bold and underlined represent ring formation; |
| | | wherein Aib represents 2-Aminoisobutyric acid |
| 22 | Oxyntomodulin | HAibQGTFTSDYS K YLD E KRAKEFVQWLMNT; wherein amino |
| | Analog | 2 acids in bold and underlined represent ring formation; |
| | | wherein Aib represents 2-Aminoisobutyric acid |
| 23 | Oxyntomodulin | His-(Aib)-Gln-Gly-Thr-Phe-Thr-Ser-Asp-Tyr-Ser-Lys-Tyr-Leu- |
| | Analog 3 | Asp-Ser-Lys-Lys-Ala-Gln-Glu-Phe-Val-Gln-Trp-Leu-Leu-Asn- |
| | | (Aib)-Gly-Arg-Asn-Arg-Asn-Asn-Ile-Ala-Xaa₃₈-Xaa₃₉ |
| | | wherein Xaa₃₈ is Cys, Cys-PEG, or is absent; Xaa₃₉ is Cys, or |
| | | is absent; and wherein the C-terminal amino acid is |
| | | optionally amidated. |
| 24 | GLP-1/GIP | Y X₂ X₃ GTFTSD X₁₀ S X₁₂ X₁₃ X₁₄ X₁₅ X₁₆ X₁₇ X₁₈ X₁₉ X₂₀ X₂₁ F |
| | Dual Agonist | X₂₃ X₂₄ W L X₂₇ X₂₈ X₂₉ X₃₀; |
| | | wherein X₂ is S or Aib; X₃ is E or Q; X₁₀ is Y, V, or L; X₁₂ is |
| | | K, S, or I; X₁₃ is L, Y, or A; X₁₄ is L or M; X₁₅ is E or D; X₁₆ |
| | | is E, G, or K; X₁₇ is E, Q, or I; X₁₈ is A or H; X₁₉ is V, A, or |
| | | Q; X₂₀ is R, K, or Q; X₂₁ is L, E, or D; X₂₃ is I or V; X₂₄ is |
| | | E, A, or N; X₂₇ is L, K, or V; X₂₈ is A, K, or N; X₂₉ is G or Q; |
| | | and X₃₉ is no amino acid, G, N, P, K, or T. |
| 25 | GLP- | HAibQGTFTSDY SKYLDEQAAK EFICWLMNT-NH2; wherein |
| | 1/Glucagon | amino acids in bold and underlined represent ring formation; |
| | Dual Agonist | wherein Aib represents 2-Aminoisobutyric acid |
| 26 | PTH | SVSEI QLMHN LGKHL NSMER VEWLR KKLQD VHNF |
| 27 | PTHrP Analog | AVSEH QLLHD KGSIQ NDLRR RELLE KLLAibK LHTA; wherein |
| | | Aib represents 2-Aminoisobutyric acid |
| 28 | F598-IgG1 LC | QLVLTQSPSA SASLGASVKL TCTLSSGHSN YAIAWHQQQP |
| | | GKGPRYLNKV NRDGSHIRGD GIPDRFSGST SGAERYLTIS |
| | | SLQSEDEADY YCQTWGAGIR VFGGGTKLTV |
| | | LGAAPSVFIFPPSDEQLKSGTASVVCLLNNFYPREAKVQWKVDNA |
| | | LQSGNSQESVTEQDSKDSTYSLSSTLTLSKADYEKHKVYACEVT |
| | | HQGLSSPVTKSFNRGEC |
| 29 | F598-IgG1 HC | QVQLQKSGPGLVKPSETLSLTCTVSGGSISGYYWSWIRQPPGKG |
| | | LEWIGYIHYSRSTNSN |
| | | PALKSRVTISSDTSKNQLSLRLSSVTAADTAVYYCARDTYYYDSG |
| | | DYEDA |
| | | FDIWGQGTMVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKKVEPPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVD |
| | | KSRWQQGNVFSCSVMHEALHNHYTQKSLSLSPGK |
| 30 | F598-IgG1 | QVQLQKSGPGLVKPSETLSLTCTVSGGSISGYYWSWIRQPPGKG |
| | HC-HSA | LEWIGYIHYSRSTNSN |
| | | PALKSRVTISSDTSKNQLSLRLSSVTAADTAVYYCARDTYYYDSG |
| | | DYEDA |
| | | FDIWGQGTMVTVSSASTKGPSVFPLAPSSKSTSGGTAALGCLVK |
| | | DYFPEPVTVSWNSGALTSGVHTFPAVLQSSGLYSLSSVVTVPSS |
| | | SLGTQTYICNVNHKPSNTKVDKKVEPPKSCDKTHTCPPCPAPELL |
| | | GGPSVFLFPPKPKDTLMISRTPEVTCVVVDVSHEDPEVKFNWYV |
| | | DGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVD |
| | | KSRWQQGNVFSCSVMHEALHNHYTQKSLSLSPGK(GGGGS)ₙDA |
| | | HKSEVAHRFKDLGEENFKALVLIAFAQYLQQCPFEDHVKLVNEVT |
| | | EFAKTCVADESAENCDKSLHTLFGDKLCTVATLRETYGEMADCC |
| | | AKQEPERNECFLQHKDDNPNLPRLVRPEVDVMCTAFHDNEETFL |
| | | KKYLYEIARRHPYFYAPELLFFAKRYKAAFTECCQAADKAACLLPK |
| | | LDELRDEGKASSAKQRLKCASLQKFGERAFKAWAVARLSQRFPK |
| | | AEFAEVSKLVTDLTKVHTECCHGDLLECADDRADLAKYICENQDS |
| | | ISSKLKECCEKPLLEKSHCIAEVENDEMPADLPSLAADFVESKDVC |
| | | KNYAEAKDVFLGMFLYEYARRHPDYSVVLLLRLAKTYETTLEKCC |
| | | AAADPHECYAKVFDEFKPLVEEPQNLIKQNCELFEQLGEYKFQNA |
| | | LLVRYTKKVPQVSTPTLVEVSRNLGKVGSKCCKHPEAKRMPCAE |
| | | DYLSVVLNQLCVLHEKTPVSDRVTKCCTESLVNRRPCFSALEVDE |
| | | TYVPKEFNAETFTFHADICTLSEKERQIKKQTALVELVKHKPKATK |
| | | EQLKAVMDDFAAFVEKCCKADDKETCFAEEGKKLVAASQAALGL; |
| | | wherein n = 0, 1, 2, 3, or 4 |
| 31 | Denosumab | EIVLTQSPGTLSLSPGERATLSCRASQSVRGRYLAWYQQKPGQA |
| | LC | PRLLIYGASSRATGIPDRFSGSGSGTDFTLTISRLEPEDFAVFYCQ |
| | | QYGSSPRTFGQGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC |
| 32 | Denosumab | EVQLLESGGGLVQPGGSLRLSCAASGFTFSSYAMSWVRQAPGK |
| | HC | GLEWVSGITGSGGSTYYADSVKGRFTISRDNSKNTLYLQMNSLR |
| | | AEDTAVYYCAKDPGTTVIMSWFDPWGQGTLVTVSSASTKGPSVF |
| | | PLAPCSRSTSESTAALGCLVKDYFPEPVTVSWNSGALTSGVHTF |
| | | PAVLQSSGLYSLSSVVTVPSSNFGTQTYTCNVDHKPSNTKVDKT |
| | | VERKCCVECPPCPAPPVAGPSVFLFPPKPKDTLMISRTPEVTCVV |
| | | VDVSHEDPEVQFNWYVDGVEVHNAKTKPREEQFNSTFRVVSVLT |
| | | VVHQDWLNGKEYKCKVSNKGLPAPIEKTISKTKGQPREPQVYTLP |
| | | PSREEMTKNQVSLTCLVKGFYPSDIAVEWESNGQPENNYKTTPP |
| | | MLDSDGSFFLYSKLTVDKSRWQQGNVFSCSVMHEALHNHYTQK |
| | | SLSLSPGK |
| 33 | Denosumab | EVQLLESGGGLVQPGGSLRLSCAASGFTFSSYAMSWVRQAPGK |
| | HC-HSA | GLEWVSGITGSGGSTYYADSVKGRFTISRDNSKNTLYLQMNSLR |
| | | AEDTAVYYCAKDPGTTVIMSWFDPWGQGTLVTVSSASTKGPSVF |
| | | PLAPCSRSTSESTAALGCLVKDYFPEPVTVSWNSGALTSGVHTF |
| | | PAVLQSSGLYSLSSVVTVPSSNFGTQTYTCNVDHKPSNTKVDKT |
| | | VERKCCVECPPCPAPPVAGPSVFLFPPKPKDTLMISRTPEVTCVV |
| | | VDVSHEDPEVQFNWYVDGVEVHNAKTKPREEQFNSTFRVVSVLT |
| | | VVHQDWLNGKEYKCKVSNKGLPAPIEKTISKTKGQPREPQVYTLP |
| | | PSREEMTKNQVSLTCLVKGFYPSDIAVEWESNGQPENNYKTTPP |
| | | MLDSDGSFFLYSKLTVDKSRWQQGNVFSCSVMHEALHNHYTQK |
| | | SLSLSPGK(GGGGS)ₙDAHKSEVAHRFKDLGEENFKALVLIAFAQY |
| | | LQQCPFEDHVKLVNEVTEFAKTCVADESAENCDKSLHTLFGDKL |
| | | CTVATLRETYGEMADCCAKQEPERNECFLQHKDDNPNLPRLVRP |
| | | EVDVMCTAFHDNEETFLKKYLYEIARRHPYFYAPELLFFAKRYKA |
| | | AFTECCQAADKAACLLPKLDELRDEGKASSAKQRLKCASLQKFG |
| | | ERAFKAWAVARLSQRFPKAEFAEVSKLVTDLTKVHTECCHGDLL |
| | | ECADDRADLAKYICENQDSISSKLKECCEKPLLEKSHCIAEVENDE |
| | | MPADLPSLAADFVESKDVCKNYAEAKDVFLGMFLYEYARRHPDY |
| | | SVVLLLRLAKTYETTLEKCCAAADPHECYAKVFDEFKPLVEEPQN |
| | | LIKQNCELFEQLGEYKFQNALLVRYTKKVPQVSTPTLVEVSRNLG |
| | | KVGSKCCKHPEAKRMPCAEDYLSVVLNQLCVLHEKTPVSDRVTK |
| | | CCTESLVNRRPCFSALEVDETYVPKEFNAETFTFHADICTLSEKE |
| | | RQIKKQTALVELVKHKPKATKEQLKAVMDDFAAFVEKCCKADDKE |
| | | TCFAEEGKKLVAASQAALGL; wherein n = 0, 1, 2, 3, or 4 |
| 34 | PCSK9 | ESALTQPASV SGSPGQSITI SCTGTSSDVG GYNSVSWYQQ |
| | Antibody LC | HPGKAPKLMI YEVSNRPSGV SNRFSGSKSG NTASLTISGL |
| | | QAEDEADYYC NSYTSTSMVF GGGTKLTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| 35 | PCSK9 | EVQLVQSGAE VKKPGASVKV SCKASGYTLT SYGISWVRQA |
| | Antibody HC | PGQGLEWMGW VSFYNGNTNY AQKLQGRGTM TTDPSTSTAY |
| | | MELRSLRSDD TAVYYCARGY GMDVWGQGTT VTVSSASTKG |
| | | PSVFPLAPCS RSTSESTAAL GCLVKDYFPE PVTVSWNSGA |
| | | LTSGVHTFPA VLQSSGLYSL SSVVTVPSSN FGTQTYTCNV |
| | | DHKPSNTKVD KTVERKCCVE CPPCPAPPVA GPSVFLFPPK |
| | | PKDTLMISRT PEVTCVVVDV SHEDPEVQFN WYVDGVEVHN |
| | | AKTKPREEQF NSTFRVVSVL TVVHQDWLNG KEYKCKVSNK |
| | | GLPAPIEKTI SKTKGQPREP QVYTLPPSRE EMTKNQVSLT |
| | | CLVKGFYPSD IAVEWESNGQ PENNYKTTPP MLDSDGSFFL |
| | | YSKLTVDKSR WQQGNVFSCS VMHEALHNHY TQKSLSLSPG K |
| 36 | PCSK9 | EVQLVQSGAE VKKPGASVKV SCKASGYTLT SYGISWVRQA |
| | Antibody HC- | PGQGLEWMGW VSFYNGNTNY AQKLQGRGTM TTDPSTSTAY |
| | HSA | MELRSLRSDD TAVYYCARGY GMDVWGQGTT VTVSSASTKG |
| | | PSVFPLAPCS RSTSESTAAL GCLVKDYFPE PVTVSWNSGA |
| | | LTSGVHTFPA VLQSSGLYSL SSVVTVPSSN FGTQTYTCNV |
| | | DHKPSNTKVD KTVERKCCVE CPPCPAPPVA GPSVFLFPPK |
| | | PKDTLMISRT PEVTCVVVDV SHEDPEVQFN WYVDGVEVHN |
| | | AKTKPREEQF NSTFRVVSVL TVVHQDWLNG KEYKCKVSNK |
| | | GLPAPIEKTI SKTKGQPREP QVYTLPPSRE EMTKNQVSLT |
| | | CLVKGFYPSD IAVEWESNGQ PENNYKTTPP MLDSDGSFFL |
| | | YSKLTVDKSR WQQGNVFSCS VMHEALHNHY TQKSLSLSPG |
| | | K(GGGGS)ₙDAHKSEVAHRFKDLGEENFKALVLIAFAQYLQQCPFE |
| | | DHVKLVNEVTEFAKTCVADESAENCDKSLHTLFGDKLCTVATLRE |
| | | TYGEMADCCAKQEPERNECFLQHKDDNPNLPRLVRPEVDVMCT |
| | | AFHDNEETFLKKYLYEIARRHPYFYAPELLFFAKRYKAAFTECCQA |
| | | ADKAACLLPKLDELRDEGKASSAKQRLKCASLQKFGERAFKAWA |
| | | VARLSQRFPKAEFAEVSKLVTDLTKVHTECCHGDLLECADDRADL |
| | | AKYICENQDSISSKLKECCEKPLLEKSHCIAEVENDEMPADLPSLA |
| | | ADFVESKDVCKNYAEAKDVFLGMFLYEYARRHPDYSVVLLLRLAK |
| | | TYETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTALV |
| | | ELVKHKPKATKEQLKAVMDDFAAFVEKCCKADDKETCFAEEGKK |
| | | LVAASQAALGL; wherein n = 0, 1, 2, 3, or 4 |
| 37 | Glucagon R | DIQMTQSPSSLSASVGDRVTITCRASQGIRNDLGWYQQKPGKAP |
| | Antibody LC | KRLIYAASSLQSGVPSRFSGSGSGTEFTLTISSVQPEDFVTYYCLQ |
| | | HNSNPLTFGGGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 38 | Glucagon R | QVQLVESGGGVVQPGRSLRISCAASGFTFSSYGMHWVRQAPGK |
| | Antibody HC | GLEWVAVMWYDGSNKDYVDSVKGRFTISRDNSKNTLYLQMNRL |
| | | RAEDTAVYYCAREKDHYDILTGYNYYYGLDVWGQGTTVTVSSAS |
| | | TKGPSVFPLAPCSRSTSESTAALGCLVKDYFPEPVTVSWNSGALT |
| | | SGVHTFPAVLQSSGLYSLSSVVTVPSSNFGTQTYTCNVDHKPSN |
| | | TKVDKTVERKCCVECPPCPAPPVAGPSVFLFPPKPKDTLMISRTP |
| | | EVTCVVVDVSHEDPEVQFNWYVDGVEVHNAKTKPREEQFNSTF |
| | | RVVSVLTVVHQDWLNGKEYKCKVSNKGLPAPIEKTISKTKGQPRE |
| | | PQVYTLPPSREEMTKNQVSLTCLVKGFYPSDIAVEWESNGQPEN |
| | | NYKTTPPMLDSDQSFFLYSKLTVDKSRWQQGNVFSCSVMHEAL |
| | | HNHYTQKSLSLSPKG |
| 39 | Glucagon R | QVQLVESGGGVVQPGRSLRISCAASGFTFSSYGMHWVRQAPGK |
| | Antibody HC- | GLEWVAVMVWDGSNKDYVDSVKGRFTISRDNSKNTLYLQMNRL |
| | HSA | RAEDTAVYYCAREKDHYDILTGYNYYYGLDVWGQGTTVTVSSAS |
| | | TKGPSVFPLAPCSRSTSESTAALGCLVKDYFPEPVTVSWNSGALT |
| | | SGVHTFPAVLQSSGLYSLSSVVTVPSSNFGTQTYTCNVDHKPSN |
| | | TKVDKTVERKCCVECPPCPAPPVAGPSVFLFPPKPKDTLMISRTP |
| | | EVTCVVVDVSHEDPEVQFNWYVDGVEVHNAKTKPREEQFNSTF |
| | | RVVSVLTVVHQDWLNGKEYKCKVSNKGLPAPIEKTISKTKGQPRE |
| | | PQVYTLPPSREEMTKNQVSLTCLVKGFYPSDIAVEWESNGQPEN |
| | | NYKTTPPMLDSDQSFFLYSKLTVDKSRWQQGNVFSCSVMHEAL |
| | | HNHYTQKSLSLSPKG(GGGGS)ₙDAHKSEVAHRFKDLGEENFKAL |
| | | VLIAFAQYLQQCPFEDHVKLVNEVTEFAKTCVADESAENCDKSLH |
| | | TLFGDKLCTVATLRETYGEMADCCAKQEPERNECFLQHKDDNPN |
| | | LPRLVRPEVDVMCTAFHDNEETFLKKYLYEIARRHPYFYAPELLFF |
| | | AKRYKAAFTECCQAADKAACLLPKLDELRDEGKASSAKQRLKCA |
| | | SLQKFGERAFKAWAVARLSQRFPKAEFAEVSKLVTDLTKVHTEC |
| | | CHGDLLECADDRADLAKYICENQDSISSKLKECCEKPLLEKSHCIA |
| | | EVENDEMPADLPSLAADFVESKDVCKNYAEAKDVFLGMFLYEYA |
| | | RRHPDYSVVLLLRLAKTYETTLEKCCAAADPHECYAKVFDEFKPL |
| | | VEEPQNLIKQNCELFEQLGEYKFQNALLVRYTKKVPQVSTPTLVE |
| | | VSRNLGKVGSKCCKHPEAKRMPCAEDYLSVVLNQLCVLHEKTPV |
| | | SDRVTKCCTESLVNRRPCFSALEVDETYVPKEFNAETFTFHADIC |
| | | TLSEKERQIKKQTALVELVKHKPKATKEQLKAVMDDFAAFVEKCC |
| | | KADDKETCFAEEGKKLVAASQAALGL; wherein n = 0, 1, 2, 3, or 4 |
| 40 | GPC3 | QVQLVQSGAEVKKPGASVKVSCKASGYTFTDYEMHWVRQ |
| | Antibody HC- | APGQGLEWMGALDPKTGDTAYSQKFKGRVTLTADKSTSTA |
| | HSA | YMELSSLTSEDTAVYYCTRFYSYTYWGQGTLVTVSSASTK |
| | | GPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | GGGGSGGGGSGGGGSDAHKSEVAHRFKDLGEENFKALVL |
| | | IAFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFVE |
| | | KCCKADDKETCFAEEGKKLVAASQAALGL |
| 41 | GPC3 | QVQLVQSGAEVKKPGASVKVSCKASGYTFTDYEMHWVRQ |
| | Antibody HC- | APGQGLEWMGALDPKTGDTAYSQKFKGRVTLTADKSTSTA |
| | HSA with | YMELSSLTSEDTAVYYCTRFYSYTYWGQGTLVTVSSASTK |
| | mutations to | GPSVFPLAPSSKSTSGGTAALGCLVKDYFPEPVTVSWNSG |
| | introduce two | ALTSGVHTFPAVLQSSGLYSLSSVVTVPSSSLGTQTYICNVN |
| | additional Cys | HKPSNTKVDKKVEPKSCDKTHTCPPCPAPELLGGPSVFLFP |
| | residues in | PKPKDTLMISRTPEVTCVVVDVSHEDPEVKFNVVYVDGVEV |
| | the HSA | HNAKTKPREEQYNSTYRVVSVLTVLHQDWLNGKEYKCKVS |
| | domain | NKALPAPIEKTISKAKGQPREPQVYTLPPSREEMTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | GGGGSGGGGSGGGGSDCHKSEVAHRFKDLGEENFKALVL |
| | | IAFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFVE |
| | | KCCKADDKETCFAEEGKKLVAASQAALGLC |
| 42 | GPC3 | QVQLVQSGAEVKKPGASVKVSCKASGYTFTDYEMHWVRQ |
| | Antibody HC- | APGQGLEWMGALDPKTGDTAYSQKFKGRVTLTADKSTSTA |
| | HSA with | YMELSSLTSEDTAVYYCTRFYSYTYWGQGTLVTVSSASTK |
| | mutations to | GPSVFPLAPSSKSTSGGTAALGCLVKDYFPEPVTVSWNSG |
| | introduce two | ALTSGVHTFPAVLQSSGLYSLSSVVTVPSSSLGTQTYICNVN |
| | additional Cys | HKPSNTKVDKKVEPKSCDKTHTCPPCPAPELLGGPSVFLFP |
| | residues | PKPKDTLMISRTPEVTCVVVDVSHEDPEVKFNWYVDGVEV |
| | outside of HSA | HNAKTKPREEQYNSTYRVVSVLTVLHQDWLNGKEYKCKVS |
| | domain | NKALPAPIEKTISKAKGQPREPQVYTLPPSREEMTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | GGGGSGGGGCGGGGSDAHKSEVAHRFKDLGEENFKALVL |
| | | IAFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFVE |
| | | KCCKADDKETCFAEEGKKLVAASQAALGLGGGGDC |

TABLE 4
DNA Sequences for GPC-3 Antibody-Human Serum Albumin (HSA) Chimeric
Molecules

| SEQ | | |
|---|---|---|
| ID | | |
| NO | Name | DNA Sequence |
| 43 | GPC3 | ATGCGCCTCCCCGCTCAACTTCTTGGACTTCTCATGCTCT |
| | Antibody LC | GGGTGTCAGGCTCCTCCGGAGATGTCGTGATGACACAGT |
| | | CCCCTCTCTCACTGCCCGTGACCCCCGGCGAACCGGCTT |
| | | CTATCTCCTGCCGGTCGTCCCAATCCCTGGTGCACTCGAA |
| | | CGCCAACACTTATCTGCATTGGTACCTCCAAAAGCCAGGG |
| | | CAGAGCCCCCAGCTCCTGATCTACAAAGTGTCCAACAGGT |
| | | TCTCCGGCGTGCCGGATAGATTCTCCGGATCGGGGAGCG |
| | | GAACCGACTTCACGCTGAAGATCAGCCGCGTCGAGGCCG |
| | | AGGACGTGGGAGTGTACTACTGCAGCCAGAACACCCACG |
| | | TGCCACCGACTTTCGGACAAGGGACCAAGCTGGAGATTA |
| | | AGCGTACGGTGGCCGCCCCTTCCGTGTTTATCTTCCCGC |
| | | CCTCGGACGAACAGCTGAAGTCCGGTACTGCCTCCGTCG |
| | | TGTGCCTGCTGAACAACTTCTACCCGCGCGAGGCCAAAG |
| | | TGCAGTGGAAGGTCGATAACGCGCTGCAGTCCGGCAACA |
| | | GCCAGGAAAGCGTCACCGAACAGGACTCCAAGGACTCCA |
| | | CCTACTCGCTGAGCAGCACCTTGACTCTGTCGAAGGCCG |
| | | ACTACGAGAAGCACAAGGTCTACGCGTGCGAAGTGACCC |
| | | ATCAGGGACTGTCCTCACCTGTGACCAAGTCCTTCAATCG |
| | | GGGCGAATGTTAG |
| 44 | GPC3 | ATGGACTGGACTTGGAGAGTGTTCTGCCTTCTCGCCGTC |
| | Antibody | GCCCCCGGAGCCCATTCGCAAGTGCAGCTGGTGCAGTCC |
| | HC-HSA | GGCGCCGAAGTCAAGAAGCCCGGTGCTTCAGTGAAAGTG |
| | | TCGTGCAAGGCCTCCGGGTACACTTTCACCGATTACGAAA |
| | | TGCACTGGGTCCGCCAGGCACCGGGCCAGGGCCTGGAG |
| | | TGGATGGGCGCCCTGGACCCCAAGACTGGCGACACCGC |
| | | GTATTCGCAGAAGTTTAAGGGCCGCGTCACTCTCACTGCC |
| | | GACAAGTCCACTAGCACTGCGTACATGGAGCTGTCATCAC |
| | | TGACCAGCGAGGACACCGCCGTGTACTACTGCACTAGGT |
| | | TCTACTCATACACCTATTGGGGACAAGGCACCCTTGTCAC |
| | | CGTGTCCTCTGCTAGCACTAAGGGACCTTCGGTGTTCCCA |
| | | CTGGCCCCGTCGTCCAAGTCCACCTCGGGGGGAACCGC |
| | | GGCCCTGGGATGCCTCGTGAAGGATTACTTCCCGGAACC |
| | | TGTCACTGTGTCATGGAACAGCGGAGCACTGACCTCCGG |
| | | GGTGCACACCTTCCCCGCCGTGTTGCAGTCCAGCGGTCT |
| | | TTACTCCCTGTCCTCCGTGGTCACCGTCCCCTCCTCCTCC |
| | | CTGGGAACCCAGACTTACATCTGCAACGTCAACCACAAGC |
| | | CGTCCAACACTAAGGTCGACAAGAAGGTCGAACCCAAGT |
| | | CCTGTGACAAGACGCACACCTGTCCTCCATGCCCCGCAC |
| | | CCGAACTGCTGGGGGGTCCGTCCGTCTTTCTCTTCCCGC |
| | | CGAAGCCGAAGGACACCCTGATGATCTCAAGAACTCCAG |
| | | AAGTCACTTGCGTGGTCGTGGATGTGTCCCACGAGGATC |
| | | CCGAAGTGAAGTTCAATTGGTACGTGGACGGAGTGGAAG |
| | | TGCACAACGCCAAGACCAAGCCTCGGGAAGAACAGTACA |
| | | ACTCCACCTACCGGGTGGTGTCCGTGCTGACCGTCCTGC |
| | | ACCAAGACTGGCTGAATGGGAAGGAGTACAAGTGCAAAG |
| | | TCTCCAACAAGGCGCTGCCGGCCCCAATCGAGAAAACTA |
| | | TCAGCAAGGCCAAGGGGCAGCCCCGGGAACCTCAAGTGT |
| | | ACACCCTCCCTCCATCCCGCGAAGAAATGACCAAGAACCA |
| | | AGTGTCGCTGACTTGCCTTGTGAAGGGCTTCTACCCGTCG |
| | | GATATCGCGGTGGAATGGGAGTCCAACGGCCAGCCTGAA |
| | | AACAACTACAAGACCACTCCGCCGGTCCTTGACTCCGAC |
| | | GGCTCCTTCTTCTTGTACTCCAAGCTGACCGTGGACAAGA |
| | | GCCGGTGGCAGCAGGGCAACGTGTTCAGCTGCAGCGTG |
| | | ATGCACGAGGCCCTGCATAACCACTATACCCAGAAAAGCC |
| | | TGTCCCTGTCCCCTGGAAAGGGAGGGGGCGGATCGGGG |
| | | GGTGGAGGCTCCGGCGGAGGAGGATCGGACGCCCACAA |
| | | ATCAGAAGTGGCGCACCGATTCAAAGACCTGGGAGAGGA |
| | | GAACTTCAAGGCACTCGTGCTTATTGCGTTCGCTCAGTAT |
| | | CTGCAGCAGTGCCCGTTCGAGGACCACGTGAAGCTGGTC |
| | | AACGAAGTCACCGAATTCGCAAAGACCTGTGTCGCCGAT |
| | | GAAAGCGCCGAAAACTGCGACAAGTCCCTGCACACCCTG |
| | | TTCGGAGACAAGCTGTGCACTGTGGCGACTCTGCGCGAA |
| | | ACCTACGGAGAGATGGCCGACTGTTGCGCTAAGCAGGAA |
| | | CCCGAACGCAACGAGTGCTTCCTGCAACACAAGGATGAC |
| | | AATCCAAACCTCCCCCGCCTCGTGCGGCCTGAAGTCGAC |
| | | GTGATGTGCACTGCCTTCCACGACAACGAGGAAACCTTCT |
| | | TGAAGAAGTACCTGTACGAGATCGCTCGGCGGCATCCGT |
| | | ACTTCTACGCCCCGGAACTCCTGTTCTTCGCGAAGCGGTA |
| | | CAAGGCAGCCTTTACCGAGTGCTGTCAAGCCGCGGACAA |
| | | GGCCGCTTGTCTGCTGCCAAAGCTGGATGAGCTGCGGGA |
| | | TGAGGGCAAAGCCTCCTCAGCTAAGCAGCGGCTGAAGTG |
| | | CGCCAGCCTGCAGAAATTCGGCGAACGCGCCTTCAAGGC |
| | | CTGGGCTGTCGCCCGGCTGTCCCAACGGTTCCCTAAGGC |
| | | TGAGTTTGCGGAAGTGTCAAAGCTGGTCACTGACCTCACC |
| | | AAGGTGCACACTGAATGCTGTCACGGCGACCTTCTGGAG |
| | | TGCGCCGACGACAGAGCCGACCTGGCCAAGTACATTTGC |
| | | GAGAACCAGGACTCGATCTCATCCAAGCTCAAGGAGTGTT |
| | | GCGAGAAGCCGCTCCTGGAGAAAAGCCACTGCATCGCTG |
| | | AAGTGGAGAACGACGAAATGCCCGCAGACTTGCCTTCGC |
| | | TCGCCGCGGACTTCGTGGAATCTAAGGACGTGTGCAAGA |
| | | ACTATGCAGAGGCCAAGGACGTGTTTCTCGGAATGTTCCT |
| | | GTACGAATACGCGAGGCGCCACCCTGATTACTCAGTGGT |
| | | GCTGCTGCTGCGCCTCGCCAAGACCTACGAAACCACACT |
| | | GGAAAAGTGTTGTGCCGCTGCCGATCCGCACGAGTGTTA |
| | | CGCGAAGGTGTTCGATGAGTTCAAGCCGTTGGTGGAGGA |
| | | GCCGCAGAACCTGATTAAGCAGAACTGCGAACTGTTTGAA |
| | | CAGCTCGGAGAATACAAATTCCAGAACGCCCTGCTCGTG |
| | | CGGTACACTAAGAAAGTGCCGCAAGTGTCGACCCCAACC |
| | | CTGGTGGAAGTGTCGAGAAATTTGGGAAAAGTCGGATCC |
| | | AAATGCTGCAAGCATCCTGAGGCCAAGAGGATGCCTTGC |
| | | GCCGAGGATTACCTGAGCGTGGTGCTGAACCAGCTTTGC |
| | | GTGTTGCATGAAAAGACCCCCGTGTCCGACCGCGTGACT |
| | | AAGTGTTGCACCGAGTCCCTCGTGAATCGCCGGCCATGT |
| | | TTTAGCGCTCTGGAGGTGGACGAGACTTACGTGCCCAAG |
| | | GAGTTCAACGCCGAAACCTTCACCTTCCACGCCGACATCT |
| | | GCACGCTGAGCGAGAAGGAGCGGCAGATTAAGAAGCAGA |
| | | CCGCCCTCGTGGAACTCGTGAAGCATAAGCCTAAGGCCA |
| | | CCAAGGAGCAGCTGAAGGCGGTCATGGACGACTTCGCGG |
| | | CATTTGTGGAGAAGTGTTGCAAGGCCGACGACAAAGAAA |
| | | CGTGCTTCGCCGAAGAAGGAAAGAAGTTGGTGGCCGCCA |
| | | GCCAGGCCGCTCTCGGGCTGTAG |
| 45 | GPC3 | ATGGACTGGACTTGGAGAGTGTTCTGCCTTCTCGCCGTC |
| | Antibody | GCCCCCGGAGCCCATTCGCAAGTGCAGCTGGTGCAGTCC |
| | HC-HSA | GGCGCCGAAGTCAAGAAGCCCGGTGCTTCAGTGAAAGTG |
| | with | TCGTGCAAGGCCTCCGGGTACACTTTCACCGATTACGAAA |
| | mutations | TGCACTGGGTCCGCCAGGCACCGGGCCAGGGCCTGGAG |
| | | TGGATGGGCGCCCTGGACCCCAAGACTGGCGACACCGC |
| | | GTATTCGCAGAAGTTTAAGGGCCGCGTCACTCTCACTGCC |
| | | GACAAGTCCACTAGCACTGCGTACATGGAGCTGTCATCAC |
| | | TGACCAGCGAGGACACCGCCGTGTACTACTGCACTAGGT |
| | | TCTACTCATACACCTATTGGGGACAAGGCACCCTTGTCAC |
| | | CGTGTCCTCTGCTAGCACTAAGGGACCTTCGGTGTTCCCA |
| | | CTGGCCCCGTCGTCCAAGTCCACCTCGGGGGGAACCGC |
| | | GGCCCTGGGATGCCTCGTGAAGGATTACTTCCCGGAACC |
| | | TGTCACTGTGTCATGGAACAGCGGAGCACTGACCTCCGG |
| | | GGTGCACACCTTCCCCGCCGTGTTGCAGTCCAGCGGTCT |
| | | TTACTCCCTGTCCTCCGTGGTCACCGTCCCCTCCTCCTCC |
| | | CTGGGAACCCAGACTTACATCTGCAACGTCAACCACAAGC |
| | | CGTCCAACACTAAGGTCGACAAGAAGGTCGAACCCAAGT |
| | | CCTGTGACAAGACGCACACCTGTCCTCCATGCCCCGCAC |
| | | CCGAACTGCTGGGGGGTCCGTCCGTCTTTCTCTTCCCGC |
| | | CGAAGCCGAAGGACACCCTGATGATCTCAAGAACTCCAG |
| | | AAGTCACTTGCGTGGTCGTGGATGTGTCCCACGAGGATC |
| | | CCGAAGTGAAGTTCAATTGGTACGTGGACGGAGTGGAAG |
| | | TGCACAACGCCAAGACCAAGCCTCGGGAAGAACAGTACA |
| | | ACTCCACCTACCGGGTGGTGTCCGTGCTGACCGTCCTGC |
| | | ACCAAGACTGGCTGAATGGGAAGGAGTACAAGTGCAAAG |
| | | TCTCCAACAAGGCGCTGCCGGCCCCAATCGAGAAAACTA |
| | | TCAGCAAGGCCAAGGGGCAGCCCCGGGAACCTCAAGTGT |
| | | ACACCCTCCCTCCATCCCGCGAAGAAATGACCAAGAACCA |
| | | AGTGTCGCTGACTTGCCTTGTGAAGGGCTTCTACCCGTCG |
| | | GATATCGCGGTGGAATGGGAGTCCAACGGCCAGCCTGAA |
| | | AACAACTACAAGACCACTCCGCCGGTCCTTGACTCCGAC |
| | | GGCTCCTTCTTCTTGTACTCCAAGCTGACCGTGGACAAGA |
| | | GCCGGTGGCAGCAGGGCAACGTGTTCAGCTGCAGCGTG |
| | | ATGCACGAGGCCCTGCATAACCACTATACCCAGAAAAGCC |
| | | TGTCCCTGTCCCCTGGAAAGGGAGGGGGCGGATCGGGG |
| | | GGTGGAGGCTCCGGCGGAGGAGGATCGGACGCCCACAA |
| | | ATCAGAAGTGGCGCACCGATTCAAAGACCTGGGAGAGGA |
| | | GAACTTCAAGGCACTCGTGCTTATTGCGTTCGCTCAGTAT |
| | | CTGCAGCAGTGCCCGTTCGAGGACCACGTGAAGCTGGTC |
| | | AACGAAGTCACCGAATTCGCAAAGACCTGTGTCGCCGAT |
| | | GAAAGCGCCGAAAACTGCGACAAGTCCCTGCACACCCTG |
| | | TTCGGAGACAAGCTGTGCACTGTGGCGACTCTGCGCGAA |
| | | ACCTACGGAGAGATGGCCGACTGTTGCGCTAAGCAGGAA |
| | | CCCGAACGCAACGAGTGCTTCCTGCAACACAAGGATGAC |
| | | AATCCAAACCTCCCCCGCCTCGTGCGGCCTGAAGTCGAC |
| | | GTGATGTGCACTGCCTTCCACGACAACGAGGAAACCTTCT |
| | | TGAAGAAGTACCTGTACGAGATCGCTCGGCGGCATCCGT |
| | | ACTTCTACGCCCCGGAACTCCTGTTCTTCGCGAAGCGGTA |
| | | CAAGGCAGCCTTTACCGAGTGCTGTCAAGCCGCGGACAA |
| | | GGCCGCTTGTCTGCTGCCAAAGCTGGATGAGCTGCGGGA |
| | | TGAGGGCAAAGCCTCCTCAGCTAAGCAGCGGCTGAAGTG |
| | | CGCCAGCCTGCAGAAATTCGGCGAACGCGCCTTCAAGGC |
| | | CTGGGCTGTCGCCCGGCTGTCCCAACGGTTCCCTAAGGC |
| | | TGAGTTTGCGGAAGTGTCAAAGCTGGTCACTGACCTCACC |
| | | AAGGTGCACACTGAATGCTGTCACGGCGACCTTCTGGAG |
| | | TGCGCCGACGACAGAGCCGACCTGGCCAAGTACATTTGC |
| | | GAGAACCAGGACTCGATCTCATCCAAGCTCAAGGAGTGTT |
| | | GCGAGAAGCCGCTCCTGGAGAAAAGCCACTGCATCGCTG |
| | | AAGTGGAGAACGACGAAATGCCCGCAGACTTGCCTTCGC |
| | | TCGCCGCGGACTTCGTGGAATCTAAGGACGTGTGCAAGA |
| | | ACTATGCAGAGGCCAAGGACGTGTTTCTCGGAATGTTCCT |
| | | GTACGAATACGCGAGGCGCCACCCTGATTACTCAGTGGT |
| | | GCTGCTGCTGCGCCTCGCCAAGACCTACGAAACCACACT |
| | | GGAAAAGTGTTGTGCCGCTGCCGATCCGCACGAGTGTTA |
| | | CGCGAAGGTGTTCGATGAGTTCAAGCCGTTGGTGGAGGA |
| | | GCCGCAGAACCTGATTAAGCAGAACTGCGAACTGTTTGAA |
| | | CAGCTCGGAGAATACAAATTCCAGAACGCCCTGCTCGTG |
| | | CGGTACACTAAGAAAGTGCCGCAAGTGTCGACCCCAACC |
| | | CTGGTGGAAGTGTCGAGAAATTTGGGAAAAGTCGGATCC |
| | | AAATGCTGCAAGCATCCTGAGGCCAAGAGGATGCCTTGC |
| | | GCCGAGGATTACCTGAGCGTGGTGCTGAACCAGCTTTGC |
| | | GTGTTGCATGAAAAGACCCCCGTGTCCGACCGCGTGACT |
| | | AAGTGTTGCACCGAGTCCCTCGTGAATCGCCGGCCATGT |
| | | TTTAGCGCTCTGGAGGTGGACGAGACTTACGTGCCCAAG |
| | | GAGTTCAACGCCGAAACCTTCACCTTCCACGCCGACATCT |
| | | GCACGCTGAGCGAGAAGGAGCGGCAGATTAAGAAGCAGA |
| | | CCGCCCTCGTGGAACTCGTGAAGCATAAGCCTAAGGCCA |
| | | CCAAGGAGCAGCTGAAGGCGGTCATGGACGACTTCGCGG |
| | | CATTTGTGGAGAAGTGTTGCAAGGCCGACGACAAAGAAA |
| | | CGTGCTTCGCCGAAGAAGGAAAGAAGTTGGTGGCCGCCA |
| | | GCCAGGCCGCTCTCGGGCTGTAG |

TABLE 5
Amino acid sequences of CDRs of Trop-2 antibody

| SEQ | | |
|---|---|---|
| ID | | |
| NO | Name | Amino Sequence |
| 46 | HCDR1 | NYGMN |
| 47 | HCDR2 | WINTYTGEPTYTDDFKG |
| 48 | HCDR3 | GGFGSSYWYFDV |
| 59 | LCDR1 | KASQDVSIAVA |
| 50 | LCDR2 | SASYRYT |
| 51 | LCDR3 | QQHYITPLT |

TABLE 6
Amino acid sequences of CDRs of Mesothelin-
binding scFv or antibody

| SEQ | | |
|---|---|---|
| ID | | |
| NO | Name | Amino Sequence |
| 52 | HCDR1 | GYTMN |
| 53 | HCDR2 | LITPYNGASSYNQKFRG |
| 54 | HCDR3 | GGYDGRGFDY |
| 55 | LCDR1 | SASSSVSYMH |
| 56 | LCDR2 | DTSKLAS |
| 57 | LCDR3 | QQWSGYPLT |

### EXAMPLES

### Example 1—Production of GPC3 Antibody, the Chimeric Molecules Comprising GPC3 Antibody and Albumin

GPC3 antibody and GPC3 Antibody-Albumin Chimeric molecules were expressed through transient expression by HEK-293 cells. Briefly, DNAs were synthesized by DND 2.0 for expressing the light chain (with amino acid sequence as shown in SEQ ID NO:10), the heavy chain (with amino acid sequence as shown in SEQ ID NO:11), the heavy chain-albumin fusion protein (with amino acid sequence as shown in SEQ ID NO:41), and the heavy chain-albumin fusion protein with two point mutations in the albumin domain (with amino acid sequence as shown in SEQ ID NO:42). The antibody-albumin chimeric molecule with the light chain of SEQ ID NO:10 and the heavy chain-albumin fusion protein of SEQ ID NO:41 was named Anti-GPC3-AB#1. The antibody-albumin chimeric molecule with the light chain of SEQ ID NO:10 and the heavy chain-albumin fusion protein of SEQ ID NO:42 was named Anti-GPC3-AB#2. The complete expression constructs with the genes of interests were confirmed by DNA sequencing. DNA constructs were transformed into E. coli DH5alfa competent cells (Invitrogen). Single clone was selected and cultured in LB broth with antibiotics (kanamycin, 25 ug/mL). DNA plasmids were extracted with Qiagen Plasmid Maxi Kit (Qiagen) following manufacture's protocol. Plasmid concentration was measured by NanoDrop (Thermo Fisher). For each molecule, the expression plasmid constructs containing the DNA sequences encoding the light chain gene and one of the heavy chain genes, were introduced into HEK-293 cells transiently by using polyethylenimine (PEI). The transfected cells were treated by alproic acid (VPA) 24 hours post transfection to enhance protein expression.

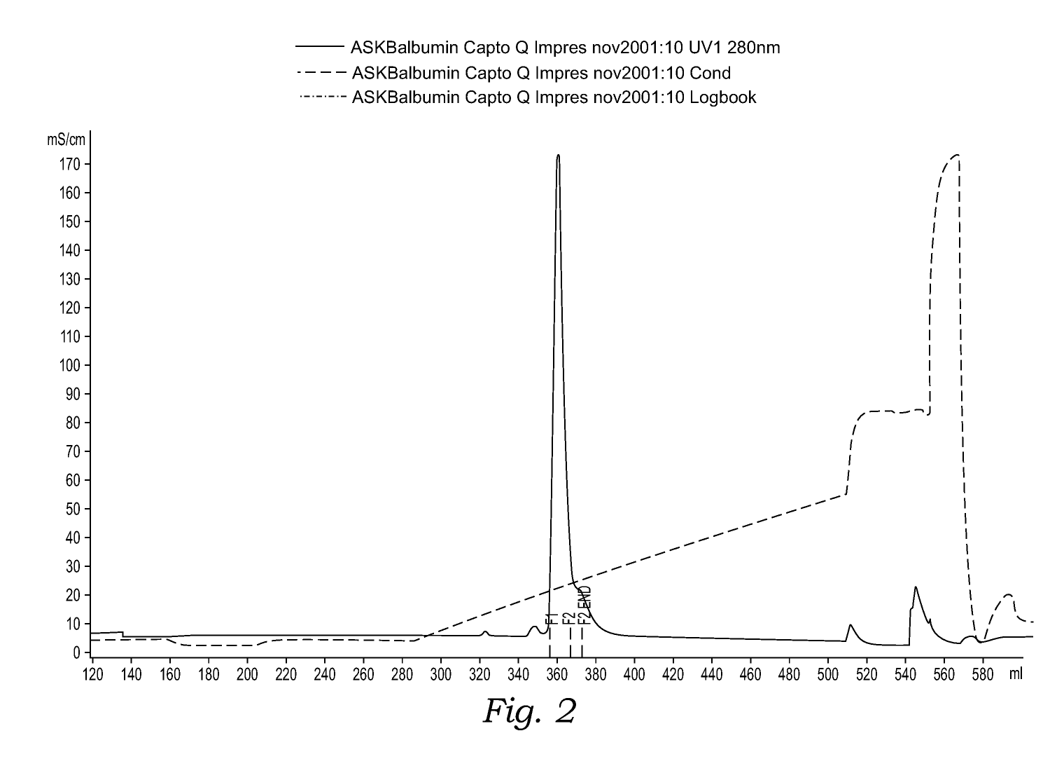

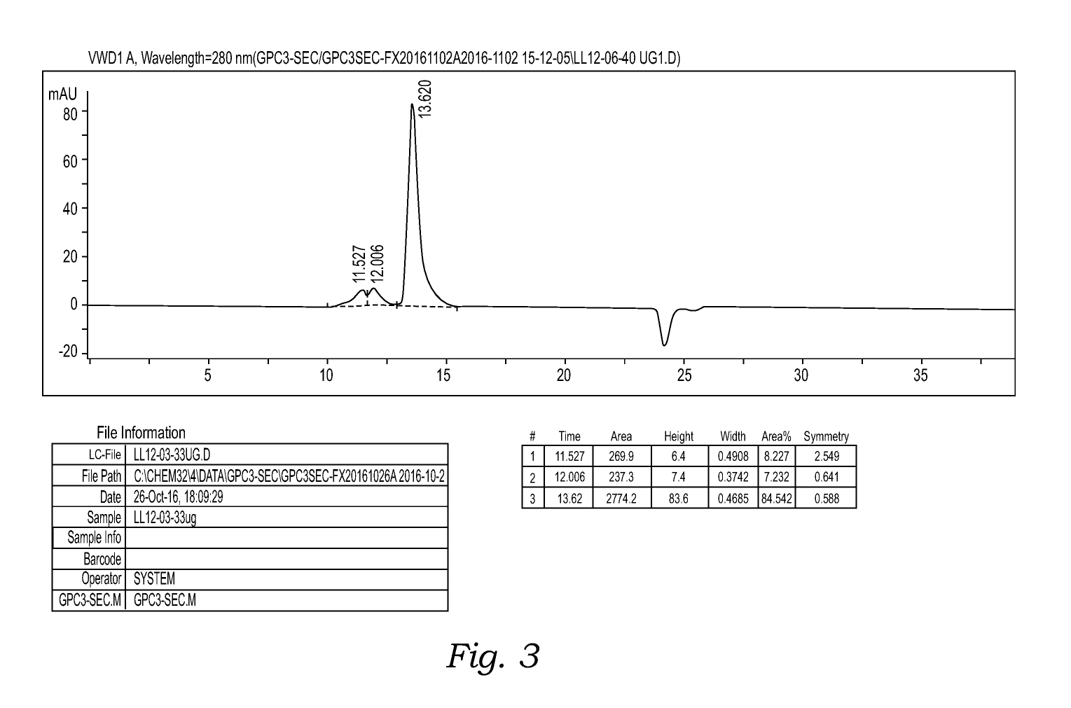

After approximately 6 days of culturing, the cell culture media were harvested by clarifying centrifugation at 9000 rpm for 30-60 minutes followed by filtration through 0.22 micrometer filters. The clarified supernants were loaded to a Protein A affinity column and the chimeric molecules were purified. The chimeric molecules were eluted using 2 M arginine solution, pH 4 from the protein A column. The chimeric molecules were further purified with additional ion exchange chromatography. FIG. 2 shows the Capto Q Impres anion exchange chromatograph for the purification of the chomeric molecule Anti-GPC3-AB#1. The purifies of the purified material were assessed by SDS-PAGE and HPLC. FIGS. 3 and 4 show the SEC-HPLC purities of the Protein A Affinity Chromatography pool and the 2^{nd} column Capto Q Impres Chromatography pool.

### Example 2. Free Thiol Measurement

In order to confirm that the cysteine residues introduced into the albumin domain of the chimeric molecule Anti-GPC3-AB#2 remained free after purification and storage, the free thiol content was measured for the purified Anti-GPC3-AB#2 sample, Lot# LL12-10, which had been stored at 2-8° C. for over 10 weeks. The free thiol measurement was carried out using the following procedure:
a. Prepare free thiol working standard (110 microM) by diluting the 110 mM free thiol standard (Thermo Fisher Scientific, Cat# M30505) 1000 times with water.
b. Further dilute the working standard to desired thiol concentrations ranged from 0-44 microM to serve as the calibration curve.
c. Obtain 10 microL of the Anti-GPC3-AB#2 sample at 0.22 mg/ml and 10 microL of a BSA solution at 2.0 mg/ml. The BSA sample is served as a positive control for this experiment. Add 40 microL of the denaturing solution (6 M Guanidine HCl) to each of the samples, and heat at 85° C. for 10 min.
d. Add 100 microL of the assay buffer to the thiol calibration curve samples and the denatured sample solutions. Heat again at 85° C. for 10 min. The assay buffer was prepared adding 60 microL of the Reagent A in the 6 mL of Reagent B, wherein both reagents A and B were provided as part of the Measure-iT Thiol Assay Kit from Thermo Fisher Scientific.
e. Place the samples and the free thiol standards into the wells of a plate and read the fluorescence at 485 nm (excitation)/528 nm (emission) using BioTeck Multi-Channel Plate Reader.
f. Calculate the free thiol based on the fluorescence readouts for the samples and for the thiol standard curve.

The results showed that the chimeric molecule Anti-GPC3-AB#2 had approximately 7 free thiols per molecule, close to the theoretical number of free thiols of 6 based on the molecule amino acid sequence. In addition, since the sample had been stored over 10 weeks at 2-8° C., the results indicated that the free thiols in the chimeric molecule appeared to be stable during the storage. The control sample BSA showed approximately 0.9 free thiol per molecule, close to the theoretical number of one free thiol per BSA molecule.

### Example 3. Conjugation of Fluorescent Dye

In order to test conjugations of the antibody-albumin chimeric molecules as well as the potential impact of albumin on the internalization process, pHAb dyes from Promega, Madison, Wis. were used. A key feature of pHAb Dyes is that they have two sulfonate groups per dye, which improve solubility in water and reduce the aggregation often seen with other non-sulfonated dyes. pHAb dye is a pH sensor florescence dye that has very low florescence at pH>7 but as pH become acidic, even after the dye conjugated to antibody. Any protein containing primary amines on lysine amino acids or thiols on the cysteine amino acids can be conjugated with pHAb Dyes. Conjugating pHAb Reactive Dyes to Antibodies is easy to detect after internalization because of the low pH environment inside the cells. The dye florescence cannot be detected when on cell surface

Conjugations to the primary amines of the GPC3 antibodies as well as the chimeric molecules Anti-GPC3-AB#1 and Anti-GPC3-AB#2. Conjugations to the free thiols of the chimeric molecules Anti-GPC3-AB#1 and Anti-GPC3-AB#2 have also been carried out. For example, conjugations to the free thiols of the chimeric molecules Anti-GPC3-AB#2 was carried out as the following:

### Materials:

Human GPC3 antibody-albumin chimeric molecule Anti-GPC3-AB#2, Lot# LL12-10, 0.22 mg/ml. pHAb Thiol reactive dye kit (Promega cat# G9831).

### Experimental Procedure

a. Exchanged buffer of the chimeric molecule into the 10 mM phosphate buffer pH7.0, which contained 1 mM EDTA and 2 M urea. Briefly, a 30 KD molecular weight cut-off (MWCO) Centrifugal Filter unit from Millipore was used for the buffer exchange and concentrating of the antibody solution. The protein concentration was adjusted to about 2 mg/ml.
b. Quickly centrifuged the pHAb Thiol dye (14000 rpm for 1 min.) in an Eppendorf tube. Added 25 micoL of DMSO: water (1:1) solution to the tube and vortexed for 1-3 min.
c. Added 12 microL of the dye solution to every 1 mg of the GPC3 antibody-albumin chimeric molecule solution, and incubated overnight while mixing gently.
d. Removed the unreactive dye with the centrifugal filter unit (MWCO of 30KD) with buffer exchanging with the 10 mM phosphate buffer (pH 7.0) with 1 mM EDTA and 2 M urea three times.
e. Measured the absorption of the solution at 280 nm and 532 nm. The dye to antibody ratio (similar to DAR in ADC) can be estimated using the formula below:

Dye-to-Antibody Ratio(DAR)=(A532×Mab_MW)/Ab Concentration(mg/ml)×75,000

### Note:

Molecular weight of GPC-3-2AB-#2=280,543

Extinction coefficient of pHAb Reactive Dye=75,000

A280 is the measured absorption at 280 nm; A532 is the measured absorption at 532 nm.

The theoretical extinction coefficient for Anti-GPC3-AB#2 is 135270

Abs 0.1% (or 1 g/1)=0.964, assuming all pairs of Cys residues form cystines

Per the protocol provided by the vendor, the correction factor for the pHAb Reactive Dye=0.256

Antibody concentration(mg/ml)=[A280−(A532×0.256)]/0.964

### Results:

5.5 ml of the Anti-GPC3-AB#2 solution (Lot# LL12-10) was used for the labeling experiment. It yielded approximately 0.25 ml solution after the labeling and buffer exchanging. The absorption was measured at both 280 nm and 532 nm:

A280=4.5

A532=4.6

Anti-GPC3-AB#2Concentration(mg/ml)=[A280−(A532×0.256)]/0.964=(4.5-4.6×0.256)/0.964=3.44 mg/ml

Dye-to-Antibody Ratio(DAR)=(A532×280,000)/[Ab Concentration(mg/ml)×75,000]=4.6×280,574/(3.44×75,000)=5.00

In summary, the GPC-3 antibody-albumin chimeric molecule Anti-GPC3-AB#2 was able to be labeled through thiol group. It was able to reach a dye to antibody ratio of approximately 5. Note that the free thiol measurement above showed that there were approximately 7 free thiols per chimeric molecule.

### Example 4. Internalization Assay

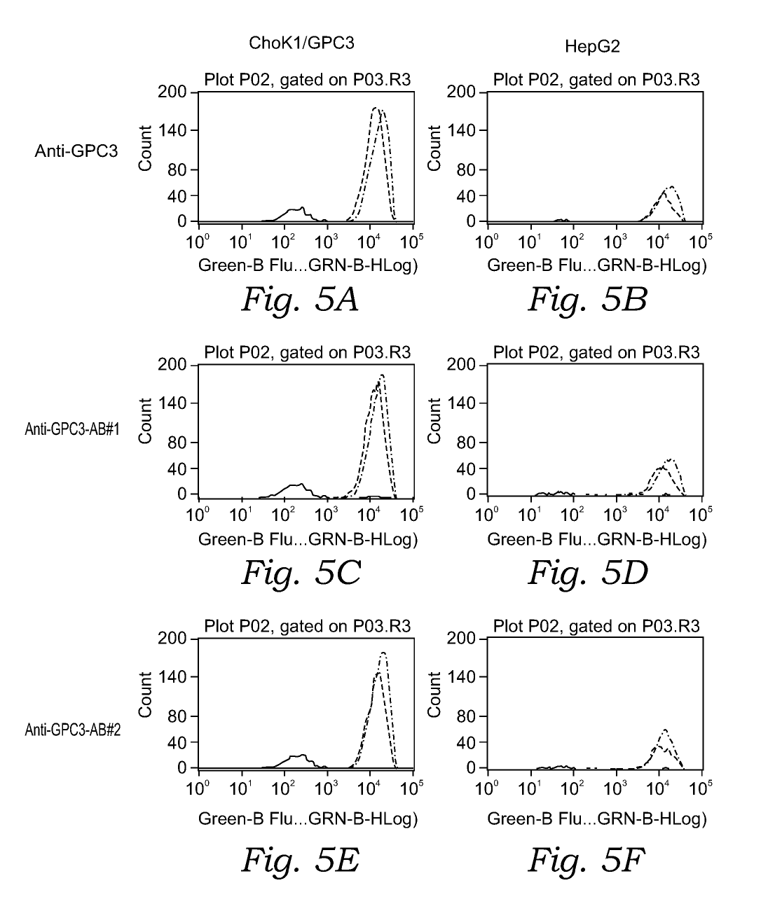

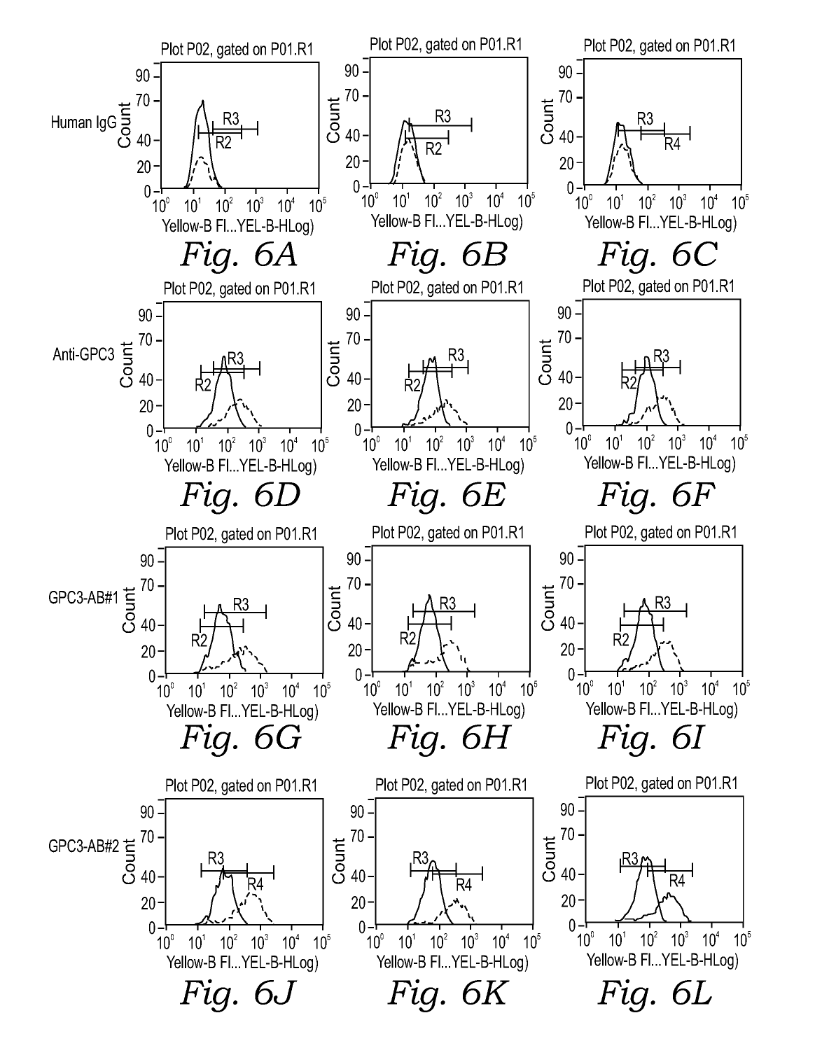

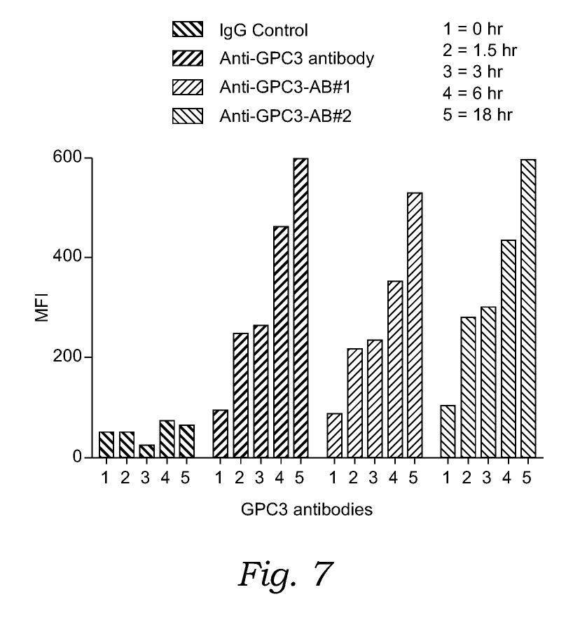

The expression of GPC-3 on the HEK293 cells transfected with GPC-3 and the HepG2 cancer cells was detected with the anti-GPC3 antibody as well as the Anti-GPC3-albumin chimeric molecules (FIG. 5). The internalization of the GPC-3 antibody and the antibody-albumin chimeric molecules were tested by FACS. HepG2 cells expressing GPC3 were cultured in RPMI 1640 medium with L-glut, 10% FBS, ×1 NEAA, 1×Pyruvate, Pen/Strep, 45 microM beta-mercaptoethanol. When reaching up to 70-90% confluency, the cells were detached with non-enzymed detachment solution and counted. 0.25 million cells (50 microL) were added in FACS buffer (3% human serum in PBS). The cells were seeded into two 96 well plates (Plate #1 and Plate#2). Added human Fc blocker with 1:50× dilution to both plates and cultured cells for 30 min on ice. The GPC3 antibody and the antibody-albumin chimeric molecules were conjugated with the pHAb dye. The antibody and the chimeric molecules were added to the cells at a final concentration of 10 microg/ml and incubated for 1 hr on ice. After that, one plate was for kept on ice, another one at 37° C. The cells were washed three times with FACS buffer and the fluorescence was read with Guava easyCyte Yellow B (583/26 nM) at different time points, including 0 hr, 1.5 hr, 3 hr, 6 hr, and 18 hr for both on ice and at 37° C. The results are shown in FIGS. 6 and 7.

### Example 5. Conjugation of MMAE to the GPC3 Antibody-Albumin Chimeric Molecules

Drug conjugation to free thiol side chains on monoclonal antibodies is a well-established technology. For example, Law et al. (Clin Cancer Res. 2004 Dec. 1; 10(23):7842-51) described the conjugation of Auristatin to Anti-CD20 antibody. Similar method can be used to conjugate drug molecules to the free thiols in the albumin domains in the chimeric molecule. Briefly, the free cysteines of the albumin domains of the antibody-albumin chimeric molecule are conjugated to antimitotic agent monomethyl auristatin E (MMAE) cytotoxins via a maleimido linker for a minimum of 30 minutes at room temperature. The reaction is then quenched with the addition of 1.2 molar excess of N-acetyl-cysteine (NAC) using a 10 mM stock solution prepared in water. After a minimum quench time of 20 minutes, the pH is adjusted to 6.0 with the addition of 0.5 M acetic acid. The various conjugated preparations of antibody-albumin and MMAE are then buffer exchanged into 20 mM histidine chloride pH 6.0 by diafiltration using a 30 kDa membrane. The final antibody-drug preparations are sterile filtered, and stored frozen.

The concentration of antibody-drug conjugates can be determined by UV absorbance. The drug/Ab ratios, the level of free drug, and the level of aggregation are determined by peptide mapping, reverse phase-HPLC and SEC-HPLC.

### Example 6. In Vitro Cytotoxicity of the Conjugates

Assays are run to demonstrate the ability of above said conjugates to effectively kill cells expressing the human GPC3 antigen in vitro. In this regard the assay measures the ability of the conjugate to kill the liver cancer cell HepG2 cells, which naturally express GPC3. In this assay killing requires binding of the ADC to its GPC3 target on the cell surface followed by internalization of ADC. Upon internalization the linker is cleaved and releases the MMAE toxin inside the cells leading to cell death. Cell death is measured using CELL TITER GLO® (Promega) reagent that measures ATP content as a surrogate for cell viability.

Specifically, 500 cells per well in DMEM supplemented with 10% fetal bovine serum and penicillin/streptomycin (DMEM complete media), were plated into 96 well tissue culture treated plates one day before the addition of antibody drug conjugates. 24 hours post plating cells are treated with serially diluted of the chimeric molecule as well as the conjugate in DMEM complete media. The cells were cultured for 96 hours post treatment, after which, viable cell numbers are enumerated using CELL TITER GLO® (Promega) as per manufacturer's instructions.

### Example 7. In Vivo Tumor Growth Suppression Study

SCID mice age of approximately 6 weeks are used in this in vivo efficacy study. The animal experiment is approved by the Ethics Committee of the animal facility, and is carried out in accordance with the Guiding Principles on the Care and Use of Animals of the facility. All procedures are performed under sodium pentobarbital anesthesia, and all efforts are made to minimize suffering. HepG2 cells are maintained at exponential growth of prior to collection. The cells are collected by trypsinizing the cells. Cell count concentration and viability is determined with trypan blue (min 98% viability). Cell suspensions are then adjusted to the required concentration for inoculation. One million cells, in a volume of 100 uL (matrigel+HepG2 suspension), is inoculated subcutaneously (s.c) via a single injection into the back of the animals. Tumor volumes and mouse weights are monitored twice weekly. When tumor volumes reach 100 mm³, mice are randomly assigned to treatment groups and injected with doses of the above said conjugates, antibody-albumin chimeric molecule or buffer control via intraperitoneal injection once every two days. There are a total of 15 injections, and the treatments last for 30 days. Following treatment, tumor volumes and mouse weights are monitored until tumors exceed 2000 mm³ or mice become sick. The animals are then euthanized. Tumors are removed, their weights logged and the tumor is documented (digital imaging).

The non-limiting examples above are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating disease.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. An isolated antibody-albumin fusion molecule, comprising an antibody and at least one albumin motif, wherein the albumin motif is fused to the heavy chain and/or light chain of said antibody, optionally through a peptide linker, wherein the albumin motif is a human serum albumin variant, which is a mutant of human serum albumin, which has been mutated such that the mutant contains a total of two or more unpaired cysteine residues, and wherein the albumin variant has at least 90% sequence identity to the amino acid sequence of SEQ ID NO:1.

2. The isolated fusion molecule of claim 1, wherein
a. each albumin motif contains one or more substitutions of non-cysteine residue to cysteine residue at a position selected from the group consisting of L585, D1, A2, D562, A364, A504, E505, T79, E86, D129, D549, A581, D121, E82, S270, Q397 and A578 of SEQ ID NO:1; and/or
b. each albumin motif contains one or more insertions of a cysteine residue at a position adjacent to the N- or C-side of an amino acid at a position selected from the group consisting of L585, D1, A2, D562, A364, A504, E505, T79, E86, D129, D549, A581, D121, E82, S270, Q397 and A578 of SEQ ID NO:1; and/or
c. the albumin variant contains of one or more free thiol groups at a position selected from the group consisting of C369, C361, C91, C177, C567, C316, C75, C169, C124 and C558, which are generated by deletion or substitution of C360, C316, C75, C168, C558, C361, C91, C124, C169 and C567.

3. An isolated chimeric molecule, which comprises the fusion molecule of claim 1, which further comprises at least two antibiotic molecules, wherein the antibiotic molecules are conjugated to the unpaired cysteine residues of the albumin motif, optionally through a linker; wherein the antibody binds to one or more antigens on the surface of a bacterium or a bacterium with multidrug resistance.

4. The chimeric molecule of claim 3, wherein the antibody binds to an antigen or antigens selected from the group consisting of CifA, ABC Transporter, Lipoteioic Acid, Iron Surface Determinant B, and Poly-N-Acetyl-Glucosamine (PNAG); wherein the antibody is selected from the group consisting of F598, Aurexis, Aurograb, and Pagibaximab.

5. The chimeric molecule of claim 3, wherein the drug molecule is an antibiotic selected from the group consisting of daptomycin, Trimethoprim/sulfamethoxazole (TMP/SMX), vancomycin, Linezolid, Quinupristin/dalfopristin, and Ceftarolin.

6. An isolated chimeric molecule, which comprises the fusion molecule of claim 1, which further comprises at least two cytotoxic drug molecules, wherein the drug molecules are conjugated to the unpaired cysteine residues of the albumin motif, preferably through a linker; wherein the antibody binds to one or more antigens on a cancer cell.

7. An isolated chimeric molecule, which comprises an antibody, at least one albumin or albumin fragment, and at least one cytotoxic drug molecule, an active peptide or an antibiotic, wherein the drug molecule, peptide or antibiotics is conjugated to the albumin or albumin fragment, optionally through a linker; wherein the albumin is human serum albumin and has at least 90% sequence identity to the amino acid sequence of SEQ ID NO:1.

8. The isolated chimeric molecule of claim 6, wherein the antibody binds to an antigen selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

9. The isolated chimeric molecule of claim 7, wherein the antibody binds to an antigen selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

10. The isolated chimeric molecule of claim 6, wherein the cytotoxic drug molecule is selected from the group consisting of microtubule disrupting agents, DNA modifying agents, RNA polymerase inhibitors, and topoisomerase I inhibitors.

11. The isolated chimeric molecule of claim 7, wherein the cytotoxic drug molecule is selected from the group consisting of microtubule disrupting agents, DNA modifying agents, RNA polymerase inhibitors, and topoisomerase I inhibitors.

12. The isolated chimeric molecule of claim 10, wherein the cytotoxic drug molecule is selected from the group consisting of azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, duocarmycin, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, monomethyl auristatin E (MMAE), monomethyl auristatin F (MMAF), phenylbutyrate, prednisone, procarbazine, paclitaxel, pentostatin, pyrrolobenzodiazepine (PBD), semustine, SN-38, streptozocin, tamoxifen, taxanes, taxol, testosterone propionate, thalidomide, thioguanine, thiotepa, teniposide, topotecan, uracil mustard, vinblastine, vinorelbine, and vincristine.

13. The isolated chimeric molecule of claim 11, wherein the cytotoxic drug molecule is selected from the group consisting of azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, duocarmycin, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, monomethyl auristatin E (MMAE), monomethyl auristatin F (MMAF), phenylbutyrate, prednisone, procarbazine, paclitaxel, pentostatin, pyrrolobenzodiazepine (PBD), semustine, SN-38, streptozocin, tamoxifen, taxanes, taxol, testosterone propionate, thalidomide, thioguanine, thiotepa, teniposide, topotecan, uracil mustard, vinblastine, vinorelbine, and vincristine.

14. The isolated chimeric molecule of claim 6, wherein the antibody binds to one or more antigens on a cancer cell, and wherein the chimeric molecule is internalized upon the binding of the chimeric molecule to the antigen.

15. The isolated chimeric molecule of claim 7, wherein the antibody binds to one or more antigens on a cancer cell, and wherein the chimeric molecule is internalized upon the binding of the chimeric molecule to the antigen.

16. The isolated chimeric molecule of claim 6 wherein the antibody is selected from the group consisting of:
a. Trastuzumab or a HER2 antibody, which comprises a light chain having an amino acid sequence with at least 98%, 99% or 100% identity to SEQ ID NO:2 and a heavy chain having an amino acid sequence with at least 95%, 96%, 97%, 98%, 99% or 100% identity to SEQ ID NO:3;
b. Rutuximab or a CD20 antibody, which comprises a light chain having an amino acid sequence with at least 98%, 99% or 100% identity to SEQ ID NO:4 and which comprises a heavy chain having an amino acid sequence with at least 95%, 96%, 97%, 98%, 99% or 100% identity to SEQ ID NO:5;
c. Brentuximab or a CD30 antibody, which comprises a light chain having an amino acid sequence with at least 98%, 99% or 100% identity to SEQ ID NO:10 and which comprises a heavy chain having an amino acid sequence with at least 95%, 96%, 97%, 98%, 99% or 100% identity to SEQ ID NO:11;
d. Cetuximab or an EGFR antibody, which comprises light chains with amino acid sequence at least 98%, 99% or 100% identical to SEQ ID NO:6 and heavy chains with amino acid sequence at least 95%, 96%, 97%, 98%, 99% or 100% identical to SEQ ID NO:7;
e. Panitumumab or an EGFR antibody, which comprises light chains with amino acid sequence at least 98%, 99% or 100% identical to SEQ ID NO:8 and heavy chains with amino acid sequence at least 95%, 96%, 97%, 98%, 99% or 100% identical to SEQ ID NO:9;
f. A DLL-3 antibody which binds to the EGF domain of the DLL-3 molecule;
g. DLL-3 antibody which binds to the DSL domain of the DLL-3 molecule;
h. DLL-3 antibodies DL301, DL302, DL305, DL306, DL308, DL309, and DL312, and their humanized versions;
i. A C-MET antibody which comprises a light chain having an amino acid sequence with at least 98%, 99% or 100% identity to SEQ ID NO:12 and which comprises a heavy chain having an amino acid sequence with at least 95%, 96%, 97%, 98%, 99% or 100% identity to SEQ ID NO:13;
j. A GPC-3 antibody which binds to an epitope located after the 374th amino acid of the GPC-3 molecule;
k. A GPC-3 antibody which binds to the heparin sulfate glycan of the GPC-3 molecule;
l. GPC-3 antibody GC33 and its humanized versions;
m. A GPC-3 antibody which comprises a light chain having an amino acid sequence with at least 98%, 99% or 100% identity to SEQ ID NO:14 and which comprises a heavy chain having an amino acid sequence with at least 95%, 96%, 97%, 98%, 99% or 100% identity to SEQ ID NO:15;
n. An EGFR antibody;
o. The EGFR antibody mAb806;
p. A Trop-2 antibody;
q. A trop-2 antibody which comprises the same complementarity determining regions (CDRs) as that of the humanized RS7 antibody, wherein the CDRs of the light chain variable region of the antibody or fragment thereof comprise CDR1 comprising the amino acid sequence of KASQDVSIAVA (SEQ ID NO:49); CDR2 comprising the amino acid sequence of SASYRYT (SEQ ID NO:50); and CDR3 comprising the amino acid sequence of QQHYITPLT (SEQ ID NO:51); and wherein the CDRs of the heavy chain variable region of the antibody or fragment thereof comprise CDR1 comprising the amino acid sequence of NYGMN (SEQ ID NO:46); CDR2 comprising the amino acid sequence of WINTYTGEPTYTDDFKG (SEQ ID NO:47) and CDR3 comprising the amino acid sequence of GGFGSSYWYFDV (SEQ ID NO:48);
r. A mesothelin antibody;
s. A mesothelin-binding scFv or antibody which comprises the same complementarity determining regions (CDRs) as that of SS1, wherein the CDRs of the light chain variable region of the antibody or fragment thereof comprise CDR1 comprising the amino acid sequence of SASSSVSYMH (SEQ ID NO:55); CDR2 comprising the amino acid sequence of DTSKLAS(SEQ ID NO:56); and CDR3 comprising the amino acid sequence of QQWSGYPLT (SEQ ID NO:57) and wherein the CDRs of the heavy chain variable region of the antibody or fragment thereof comprise CDR1 comprising the amino acid sequence of GYTMN (SEQ ID NO:52); CDR2 comprising the amino acid sequence of LITPYNGASSYNQKFRG (SEQ ID NO:53) and CDR3 comprising the amino acid sequence of GGYDGRGFDY(SEQ ID NO:54);
t. A Claudin 18.2 antibody which does not bind to Claudin 18.1 or binds to Claudin 18.1 with at least 10 times weaker in term of binding affinity;
u. A Claudin 18.2 antibody which comprises a light chain having an amino acid sequence with at least 98%, 99% or 100% identity to SEQ ID NO:16 and which comprises a heavy chain having an amino acid sequence with at least 95%, 96%, 97%, 98%, 99% or 100% identity to SEQ ID NO:17;
v. An antibody which binds to two different epitopes of an antigen selected from the the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6; and
w. A bispecific antibody which binds to two antigens selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, x. GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

17. The isolated chimeric molecule of claim 7 wherein the antibody is selected from the group consisting of:
a. Trastuzumab or a HER2 antibody, which comprises a light chain having an amino acid sequence with at least 98%, 99% or 100% identity to SEQ ID NO:2 and a heavy chain having an amino acid sequence with at least 95%, 96%, 97%, 98%, 99% or 100% identity to SEQ ID NO:3;
b. Rutuximab or a CD20 antibody, which comprises a light chain having an amino acid sequence with at least 98%, 99% or 100% identity to SEQ ID NO:4 and which comprises a heavy chain having an amino acid sequence with at least 95%, 96%, 97%, 98%, 99% or 100% identity to SEQ ID NO:5;
c. Brentuximab or a CD30 antibody, which comprises a light chain having an amino acid sequence with at least 98%, 99% or 100% identity to SEQ ID NO:10 and which comprises a heavy chain having an amino acid sequence with at least 95%, 96%, 97%, 98%, 99% or 100% identity to SEQ ID NO:11;
d. Cetuximab or an EGFR antibody, which comprises light chains with amino acid sequence at least 98%, 99% or 100% identical to SEQ ID NO:6 and heavy chains with amino acid sequence at least 95%, 96%, 97%, 98%, 99% or 100% identical to SEQ ID NO:7;
e. Panitumumab or an EGFR antibody, which comprises light chains with amino acid sequence at least 98%, 99% or 100% identical to SEQ ID NO:8 and heavy chains with amino acid sequence at least 95%, 96%, 97%, 98%, 99% or 100% identical to SEQ ID NO:9;
f. A DLL-3 antibody which binds to the EGF domain of the DLL-3 molecule;
g. DLL-3 antibody which binds to the DSL domain of the DLL-3 molecule;
h. DLL-3 antibodies DL301, DL302, DL305, DL306, DL308, DL309, and DL312, and their humanized versions;
i. A C-MET antibody which comprises a light chain having an amino acid sequence with at least 98%, 99% or 100% identity to SEQ ID NO:12 and which comprises a heavy chain having an amino acid sequence with at least 95%, 96%, 97%, 98%, 99% or 100% identity to SEQ ID NO:13;
j. A GPC-3 antibody which binds to an epitope located after the 374th amino acid of the GPC-3 molecule;
k. A GPC-3 antibody which binds to the heparin sulfate glycan of the GPC-3 molecule;
l. GPC-3 antibody GC33 and its humanized versions;
m. A GPC-3 antibody which comprises a light chain having an amino acid sequence with at least 98%, 99% or 100% identity to SEQ ID NO:14 and which comprises a heavy chain having an amino acid sequence with at least 95%, 96%, 97%, 98%, 99% or 100% identity to SEQ ID NO:15;
n. An EGFR antibody;
o. The EGFR antibody mAb806;
p. A Trop-2 antibody;
q. A trop-2 antibody which comprises the same complementarity determining regions (CDRs) as that of the humanized RS7 antibody, wherein the CDRs of the light chain variable region of the antibody or fragment thereof comprise CDR1 comprising the amino acid sequence of KASQDVSIAVA (SEQ ID NO:49); CDR2 comprising the amino acid sequence of SASYRYT(SEQ ID NO:50); and CDR3 comprising the amino acid sequence of QQHYITPLT (SEQ ID NO:51); and wherein the CDRs of the heavy chain variable region of the antibody or fragment thereof comprise CDR1 comprising the amino acid sequence of NYGMN (SEQ ID NO:46); CDR2 comprising the amino acid sequence of WINTYTGEPTYTDDFKG (SEQ ID NO:47) and CDR3 comprising the amino acid sequence of GGFGSSYWYFDV (SEQ ID NO:48);
r. A mesothelin antibody;
s. A mesothelin-binding scFv or antibody which comprises the same complementarity determining regions (CDRs) as that of SS1, wherein the CDRs of the light chain variable region of the antibody or fragment thereof comprise CDR1 comprising the amino acid sequence of SASSSVSYMH (SEQ ID NO:55); CDR2 comprising the amino acid sequence of DTSKLAS(SEQ ID NO:56); and CDR3 comprising the amino acid sequence of QQWSGYPLT (SEQ ID NO:57) and wherein the CDRs of the heavy chain variable region of the antibody or fragment thereof comprise CDR1 comprising the amino acid sequence of GYTMN (SEQ ID NO:52); CDR2 comprising the amino acid sequence of LITPYNGASSYNQKFRG (SEQ ID NO:53) and CDR3 comprising the amino acid sequence of GGYDGRGFDY(SEQ ID NO:54);
t. A Claudin 18.2 antibody which does not bind to Claudin 18.1 or binds to Claudin 18.1 with at least 10 times weaker in term of binding affinity;
u. A Claudin 18.2 antibody which comprises a light chain having an amino acid sequence with at least 98%, 99% or 100% identity to SEQ ID NO:16 and which comprises a heavy chain having an amino acid sequence with at least 95%, 96%, 97%, 98%, 99% or 100% identity to SEQ ID NO:17;
v. An antibody which binds to two different epitopes of an antigen selected from the the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6; and
w. A bispecific antibody which binds to two antigens selected from the group consisting of Guanyl cyclase C (GCC), carbohydrate antigen 19-9 (CA19-9), gpA33, Musin, CEA, IGF1-R, HER2, HER3, DLL-3, DLL-4, EGF Receptor or its mutants, GPC-3, C-MET, VEGF Receptor 1, VEGF Receptor 2, Nectin-4, Liv-1, x. GPNMB, PSMA, Trop-2, SC-16, CAIX, ETBR, TF, NaPi2b, STEAP1, FRalpa, SLITRK6, CA6, ENPP3, Mesothelin, 5T4, CD19, CD20, CD22, CD33, CD40, CD56, CD66e, CD70, CD74, CD79b, CD98, CD123, CD138, CD352, PD-L1, Claudin 18.2, and Claudin 6.

18. The chimeric molecule of claim 1, wherein the antibody binds to human RANK Ligand, human PCSK9, human Glucagon Receptor, and/or human ASGR1; wherein the chimeric molecule further comprises one or more active peptides, selected from 1) PTH, 2) PTHrP, 3) GLP-1 and its analogs; 3) exendin-4 and its analogs; 4) GIP and its analogs; and 5) Oxyntomodulin and its analogs.

19. The chimeric molecule of claim 7, wherein the antibody binds to human RANK Ligand, human PCSK9, human Glucagon Receptor, and/or human ASGR1; wherein the chimeric molecule further comprises one or more active peptides, selected from 1) PTH, 2) PTHrP, 3) GLP-1 and its analogs; 3) exendin-4 and its analogs; 4) GIP and its analogs; and 5) Oxyntomodulin and its analogs.

20. A nucleic acid sequence, which encodes the antibody heavy chain-albumin fusion protein or antibody light chain-albumin fusion protein of the chimeric molecule of claim 6.

21. A pharmaceutical composition comprising an isolated chimeric molecule of claim 6, and a pharmaceutically acceptable carrier.

22. A method for treating cancer in a subject, said method comprising administering to a subject in need of such a treatment using a pharmaceutical composition of claim 15.

23. The method of claim 16, wherein the cancer treatment is administered to a patient identified positive with both the antigen targeted by the said chimeric molecule, and RAS mutation, as tested by using companion diagnostic biomarker assays suitable for testing the antigen and RAS mutation.

24. The method of claim 16, wherein the cancer is colorectal cancer or pancreatic cancer.
